(19) United States
(12) Patent Application Publication
(10) Pub. No.: US 2024/0307552 A1
(43) Pub. Date: Sep. 19, 2024

(54) RNA STABILIZING NANOPARTICLES

(71) Applicant: KANSAS STATE UNIVERSITY RESEARCH FOUNDATION, Manhattan, KS (US)

(72) Inventor: Robert DeLong, Manhattan, KS (US)

(21) Appl. No.: 18/546,937

(22) PCT Filed: Feb. 18, 2022
(86) PCT No.: PCT/US2022/070739

§ 371 Date: Aug. 17, 2023

Related U.S. Application Data
(60) Provisional application No. 63/200,167, filed on Feb. 18, 2021.

(51) Int. Cl. A61K 47/69; A61K 38/17; A61K 39/215; A61K 39/385; A61K 48/00

(52) U.S. Cl. CPC A61K 47/6929; A61K 38/1729; A61K 39/215; A61K 39/385; A61K 48/0033

(57) ABSTRACT

The present disclosure provides ZnO-based compositions that stabilize mRNA and RNA as well as provide compositions and therapies to treat or prevent cancer and viral as well as microbial diseases.

### CROSS REFERENCE TO RELATED APPLICATIONS

This application relates to and claims priority to U.S. Provisional Patent Application No. 63/200,167, which was filed on Feb. 18, 2021. The teachings and contents of this reference is incorporated herein in its entirety.

### STATEMENT REGARDING FEDERALLY SPONSORED RESEARCH & DEVELOPMENT

None

### BACKGROUND OF THE DISCLOSURE

The field of the invention relates generally to nucleic acids and compositions that can stabilize the structure and/or properties of such nucleic acids.

### BRIEF DESCRIPTION OF THE DISCLOSURE

With the COVID-19 pandemic and deaths from metastatic cancer at an all-time high, the search for a biocompatible chemotherapeutic targeted nanomedicine has never been more important. Antimicrobial, antiviral and anticancer activity of zinc oxide nanoparticles (ZnO NPs) have been linked to ROS, zinc ion intracellular gradients and enzyme inhibition. Over the years a variety of different methods to synthesize nanoparticles have been developed. One early focus was on the use of gold, but gold is non-physiological and as a precious metal somewhat expensive. This focus has moved on to copper and more recently zinc-based materials knowing that in cells and tissues, zinc is often used to stabilize protein and nucleic acids and cells have evolved ways to re-use and recycle Zn, and other bio-metals like magnesium and iron. Cells also use them for a variety of reasons and other metals for example in the electron transport chain proteins or other metabolic enzymes.

Zinc oxide has been shown to have antimicrobial or antiviral activity as well as anticancer activity. In this disclosure, zinc oxide showed selectivity to both mouse and human melanoma cell lines and it showed antitumor activity in a syngeneic immuno-competent mouse melanoma model.

This disclosure also describes exploiting the protein nanobio interaction to potentiate the anticancer activity of zinc oxide. For example RAS is an oncogene commonly mutated in many aggressive highly metastatic types of cancers and even if RAS itself is not mutated its regulatory partners having RAS binding domain (RBD) or downstream effectors such as ERK and AKT pathways are active in many if not most of the worst and highly metastatic cancers including GBM, drug-resistant melanoma, small cell lung carcinoma and others. The ZnO NP interaction to RBD protein and to its delivery as a potential decoy of these pathways was investigated with promising results published in PLOS One. ZnO is weakly fluorescent in the high UV range and when the protein binds to its surface it quenches the ZnO fluorescence in a concentration manner and this could be exploited to estimate the ZnO NP protein binding constant which was substantial at 10-5 indicating its strong protein association. In one aspect, RBD is pulled down onto ZnO, eluted off, and stained in a protein gel. In another aspect, it was shown that the ZnO NP-RBD complexes induced apoptosis to melanoma cells, which is indicative of a protein decoy or interference approach whereby flooding the cell with dysfunctional RBD is basically a way to turn off RAS-based signaling. See FIG. 1

In another aspect, the present disclosure investigates the anticancer properties of ZnO and as a model cancer protein using RBD, could induce programmed cell death to melanoma cells. In some forms, the ZnO can be administered to a subject in need thereof as described herein. In some forms, the ZnO is administered systemically once or multiple times. In some forms, the ZnO is administered 1, 2, 3, 4, 5, 6, 7, 8, 9, 10, 11, 12, 13, 14, 15, or more times. In some forms, the ZnO is administered on a scheduled basis including daily, bi-daily, once every 3, 4, 5, or 6 days, weekly, bi-weekly, once every 2, 3, 4, 5, 6, 7, 8, 9, 10, 11, 12, 13, 14, 15, or more weeks, monthly, bi-monthly, every 3, 4, 5, 6, 7, 8, 9, 10, or 11 months, yearly, or any combination thereof. In some forms, the administration is via injection or infusion and any combination thereof.

In another aspect, the present disclosure describes the effect on the structure and function of biomolecules when they interact with or at a nanoparticle. Having studied Luciferase enzyme which is well-characterized and emits a highly sensitive light producing reaction we were able to measure the effects of various nanoparticles on this enzyme, both its structure and its light production. One of the interesting things we found is that iron oxide and especially copper nanoparticles had a dramatic effect on this enzyme's activity. For example, when the enzyme was complexed to copper if we incubated the complex with trypsin, which is one of the main proteases in blood or serum, the presence of the nanoparticle caused the protein to be degraded much more rapidly. After only 5 minutes of exposure not much of the original protein was present. This technique shows which parts of the protein were protected by nanoparticle interaction, i.e. which of its amino acids participated in the nanoparticle interaction, sort of nanoprotease mapping and those are indicated by the colored residues in the protein fold. When we analyzed the protein by circular dichroism (CD) spectroscopy we could see a dramatic change in its pattern helix, sheet and coil percentages dose-dependent on the copper. Basically these data indicate that copper nanoparticles destroy proteins causing them to denature and degrade more rapidly.

In another aspect of the present disclosure, the safety of ZnO NP compositions was determined.

In another aspect, a fluorescent tag was placed on ZnO NP and this nanoparticle was administered to mice or an underivatized control to check the tissue distribution and tissue and blood toxicity. The results demonstrated that the ZnO NP stabilized enzyme activity. See FIG. 3.

These mice were dosed with the 20 mg/kg chemotherapeutic dose. It was surprising to observe the nanoparticle both in the bioimager, fluorescence spectroscopy, and by ICP/Ms measuring Zn in the tissues by 3 three different techniques clearly showing NP goes not only to liver and kidney as expected, but appreciably to spleen, lung and brain, tissues which are a concern both for cancer metastasis and COVID-19 infection or where the virus can be detected. See FIG. 4.

In another aspect, we have begun to explore the zinc-based physiological metal composites or PMCs for short. In one aspect a little cobalt or nickel was incorporated onto the surface of ZnO and this appeared to increase its biocompatibility and importantly could be used to attach nucleic acids forming an amino conjugate. When incubated with cells we could clearly see the nanoparticles in the cells by optical or hyperspectral imaging, the nanoparticles are the green or light starry dots in a cell, and by putting a fluorescent tag on an antisense oligomer we could clearly see using confocal microscopy that the nanoparticles delivered ASO into cells. See FIG. 5.

In another aspect of the disclosure, the stabilization of RNA and mRNA was investigated. The rapid need for a COVID-19 vaccine has caused a resurgence in interest of mRNA vaccines and RNA-based therapeutics more generally, however their cold chain requirement greatly limits widespread deployment and efficacy. RNA interaction to zinc oxide nanoparticle (ZnO NP) increases stability in serum, liver and tumor homogenates protecting against RNase-mediated degradation and potentiates immune response in cell culture and mouse models. Here we show for the first time that RNA interaction to ZnO NP enhances RNA temperature stability. RNA interaction with or to physiological metal oxide nanoparticles was shown by zeta potential surface charge shifts for all the primary physiological metal oxide nanoparticles with the ZnO NP obtaining the highest payload (2.3 ng/mg). Interaction of RNA to ZnO NP allowed structure-function retention as shown by circular dichroism (CD). The pattern retained indeed accentuated dependent on the stoichiometric ratio of ZnO:poly I:C. Interaction of a cationic peptide, for example antiviral peptide LL-37 (SEQ ID NO. 2) could then be followed by addition of the viral RNA mimetic RNA poly I:C to form tripartite species (NP-protein:RNA) as shown by 2-dimensional fluorescence difference spectroscopy (2-D FDS) and gel electrophoresis mobility shift. Similarly protamine NP complexes could be used to bind torulayeast (TY-RNA) which imparted temperature-stabilization to either mesoporous silica nanoparticle (MSN) control or ZnO NP as shown by RNA agarose gel electrophoresis (RAGE) where the RNA could be stored unfrozen in PBS buffer yet retain a high intensity RNA stained band equivalent to control or input RNA. The ZnO-protamine-RNA dry powders precipitated from alcohol could be placed on accelerated stability protocol in controlled temperature chambers and the RNA band could be maintained for 1 to 2 days when stored at 30° C. or 40° C., but not 50° C., suggesting temperature-stability enhancement. Luciferase mRNA formulations were made and maintained at temperature in parallel and retained expression activity over time. Similarly, antigenicity experiments demonstrated stable antigenicity.

In another aspect, the ZnO NP interaction with RNA and protein and immunological potentiation was investigated. Zinc oxide nanoparticle (ZnO NP) have well-known antimicrobial, antiviral, and anticancer activity and the material is considered a nanoscale chemotherapeutic. As determined herein, ZnO NP stabilizes RNA better than DNA and protects it from RNase-A degradation and when incubated in serum, liver or tumor homogenates. However, temperature sensitivity has remained an obstacle to use. This disclosure explores RNA temperature stabilizing properties of the ZnO NP, comparatively characterized by differential scanning calorimetry (DSC), circular dichroism (CD) and accelerated stability RNA agarose gel electrophoresis (RAGE) analysis after refrigeration, room temperature exposure, 30, 40 or 50 degrees Celsius of the dried powder formulations in combination with protamine or a nanoparticle which had been previously reported to temperature stabilize RNA, mesoporous silica nanoparticle (MSN).

As is known, COVID-19 mRNA vaccine, other RNA vaccines, and macromolecular RNA therapeutics are temperature sensitive and must be stored at extremely low temperatures to maintain activity. This cold chain requirement greatly limits efficacy and widespread deployment. Inorganic nanoparticles can complex and deliver RNA and an early report suggested surface-coated mesoporous silica nanoparticle (MSN) could stabilize RNA stored at 4° C. for several days. Using torula yeast RNA (TY-RNA) obtainable in bulk in the size range typical of mRNA, we showed that RNA stability for RNA incubated at physiological temperature (37° C.) in water was in the order; ZnO>Fe₃O₄>Ag>Cu>MSN>CNT. RNA gel electrophoresis (RAGE) showed zinc oxide (ZnO—NP), iron oxide (Fe₃O₄), and silver (Ag) caused a slight gel shift consistent with their known RNA interaction, and prevented the band broadening that occurred with control RNA stored at 4° C., whereas carbon nanotube (CNT), copper (Cu), and surprisingly uncoated MSNs lost RNA band staining intensity or caused a smear demonstrating RNA degradation. RNA particle association of RNA to ZnO—NP could be increased by coating with cationic cell penetrating peptide protamine (Prot), or with antiviral LL37 peptide (SEQ ID NO. 2) with >95 to 40-50% RNA loading efficiency, respectively. Denaturation of macromolecular RNA is caused at higher temperatures, and here using an immunogenic double-stranded (dsDNA) polyinosinic:polycytidylic (poly I:C) with a defined melting temperature (Tₘ) RNA temperature-stabilization was shown by differential scanning calorimetry (DSC) with an increase in Tₘ from 64 to 71° C. upon ZnO—NP complexation. ZnO—NP RNA structural stabilization was confirmed by circular dichroism (CD) analysis with peak intensity increased as a function of ZnO:RNA stoichiometry. ZnO-Prot-RNA were placed onto accelerated stability protocols at 30, 40 and 50° C., the RNA eluted and analyzed by RAGE suggested intact RNA could be recovered at all temperatures for up to 2 weeks. In vitro translation experiments using Luciferase mRNA retained expression activity and GFP mRNA delivery in cell culture. These data support ZnO-Prot-mRNA formulations for enhanced RNA vaccine temperature stability and activity.

In another aspect, methods for increasing stability of RNA and/or mRNA are provided. In some forms, the RNA and/or mRNA are complexed or combined with a nanoparticle, preferably selected from the group consisting of ZnO, Fe₃O₄, Ag, Cu, MSN, CNT, and any combination thereof. In some forms, the increased stability is determined by a comparison of the structure of the RNA and/or mRNA with or without the complexation with the nanoparticle(s). In some forms, the stability is increased by 10, 20, 30, 40, 50, 60, 70, 80, 90, 95, 99% or more. In some forms, the increased stability is determined by a decrease in the rate and/or extent of degradation of the RNA and/or mRNA in comparison between the RNA and/or mRNA with or without the complexation with the nanoparticle(s). In some forms, the degradation is decreased by 10, 20, 30, 40, 50, 60, 70, 80, 90, 95, 99% or more. In some forms, the increased stability is determined by a comparison of the resistance to degradation of the RNA and/or mRNA with or without the complexation with the nanoparticle(s) in response to increased temperatures. In some forms, the degradation is decreased by 10, 20, 30, 40, 50, 60, 70, 80, 90, 95, 99% or more.

The success of COVID19 mRNA vaccines, RNA-guided CRISPR/Cas gene editing and clinical approval of several oligonucleotide or aptamer therapeutics has ushered in a new era of RNA-based therapy. Lipid nanoparticles (LPN) have been clinically approved, but suffer from the cold chain requirement needing to be stored at temperatures below −20° C. to retain activity. Our lab broke the DNA vaccine cold chain, temperature stabilizing the DNA by loading it into gold particles coated with protamine, a US patent was awarded for this work in 2013. In cells and tissues, zinc is perhaps best known for stabilizing RNA and protein interactions, and this disclosure focuses on the interaction of RNA to zinc oxide nanoparticles and its ability to act synergistically with RNA to induce an effective immune response. Here zinc and other inorganic nanoparticles were investigated for RNA stabilization.

In one aspect, the present disclosure provides a stabilized nucleic acid particle. In some forms, the nucleic acid particle structure and/or activity is stabilized. “Stabilized” refers to a resistance to degradation, both structurally and functionally, in comparison to a nucleic acid that has not been stabilized according to the methods described herein. In preferred forms, the nucleic acid is RNA and/or mRNA. In some forms, the nucleic acid is stabilized with an oxide, preferably in nanoparticle form. In some forms, the oxide is ZnO. In some forms, the oxide further includes cobalt or nickel on the surface thereof. In some forms, the stabilized nucleic acid can be administered to an animal, preferably a mammal, and more preferably a human. In some forms, the stabilized nucleic acid shows improved stability at warm, cold, and freezing temperatures when compared to a nucleic acid that is not combined with the nanoparticle. In some forms, the improved stability is at a temperature above −20, −15, −10, −5, 0, 5, 10, 15, 20, 25, 30, 35, 40, or 45° C.

In another aspect, the distribution, tolerance, and anticancer/antiviral activity of Zn-based physiometacomposites (PMCs) was determined. Manganese, iron, nickel and cobalt doped ZnO was synthesized. “Doped” refers to a combination of one material with another, wherein one of the two materials is smaller and/or in a lesser amount than the other material. For example, in the present disclosure, nanoparticles can be doped with a separate component, such as an element, wherein the element is both smaller in size and in a lesser amount (by weight) than the nanoparticle.” Cell uptake and distribution into 3-D culture and mice, as well as biochemical and chemotherapeutic activity were studied by fluorescence/bioluminescence, confocal microscopy, flow cytometry, viability, antitumor and virus titer assays. Luminescence and inductively coupled plasma mass spectrometry analysis showed that nanoparticle distribution was liver>spleen>kidney>lung>brain, without tissue or blood pathology. Photophysical characterization as ex vivo tissue probes and LL37 peptide (SEQ ID NO. 2), antisense oligomer (ASO) or aptamer delivery targeting RAS/RBD. 25 μg/ml 48-hour treatment showed ≥98-99% cell viability, and 3-D organoid uptake. Such data support the preclinical development of PMCs for imaging and delivery targeting cancer and infectious disease.

In another aspect, the present disclosure provides a nanoparticle. In some forms, the nanoparticle is ZnO. In some forms, the nanoparticle is doped with or combined with an element. In some forms, the element is selected from the group consisting of manganese, iron, nickel, cobalt and any combination thereof. In some forms, the nanoparticle is combined with or complexed with a protein or peptide. In some forms, the nanoparticle is combined with LL37 peptide, preferably having the sequence of SEQ ID NO. 2, an antisense oligomer (ASO), aptamer, or any combination thereof “Complexed” refers to a combination of components that can be in direct contact or indirect contact with one another. In some forms, the nanoparticle delivery targets a specific domain or organ. In some forms, the organ is selected from the group consisting of liver, spleen, kidney, lung, brain, or any combination thereof. In some forms, the domain is a particular protein segment. In some forms, the segment is RAS/RBD or a spike protein. In some forms the sequence is selected from the group consisting of SEQ ID NO. 3 or SEQ ID NO. 4.

In another aspect, the pharmacokinetics of ZnO NPs versus silica coated ZnO NPs was investigated. This disclosure demonstrates that bioavailability is better for the naked uncoated material. Previously, it was observed that zinc-based physiometacomposite (PMC) nanoparticles doped with cobalt (5% CoZnO) were biocompatible and capable of forming amino or amido-conjugates with antisense oligomer (ASO) for delivery into cells. We observed that by similarly doping magnesium into ZnO (5% MgZnO) this could red-shift its fluorescence excitation/emission. Thus, here we expanded on this chemistry to include manganese (Mn), iron (Fe), nickel (Ni) or cobalt ferrite (CoFe) doped oxide (ZnO). The ZnO-based physiologically-based metal composites, physiometacomposites (PMC) were comparatively characterized, their fluorescence, biocompatibility and delivery were investigated. Further, a few in vivo studies of ZnO NP were conducted, where cyanine 5.5-labeled ZnO (cy5.5-ZnO) and ZnO NP biodistribution was compared by fluorescence, inductively coupled plasma mass spectrometry (ICP/MS) and in vivo whole animal luminescence imaging. Anticancer activity of these and the nickel-doped zinc oxide (Ni/ZnO) was then compared against drug-resistant melanoma in conjunction with Ras binding domain (RBD) or RAS-targeted antisense or aptamer oligonucleotides. Nanoscale physiometacomposite (PMC) materials containing zinc oxide doped with manganese, iron, nickel or cobalt were synthesized.

In another aspect, this disclosure provides compositions comprising ZnO-based physiometacomposite (PMC) nanoparticles. In some forms, these PMC nanoparticles are combined or doped with cobalt, magnesium, manganese, iron, nickel, cobalt ferrite, oxide, or any combination thereof. In some forms, the material combined or doped with is present in an amount of 1, 2, 3, 4, 5, 6, 7, 8, 9, 10, or more weight percent. In some forms, the doped zinc-based PMC nanoparticles are further formed into amino or amido-conjugates. In some forms, the amino or amido-conjugates are with ASO. In some forms, any of the above PMCs can be delivered into cells. In some forms, the above PMCs are administered to a subject in need thereof. In some forms, the administration is as described herein. In some forms, the administration is systemic. In some forms, the administration is via injection or infusion. In some forms, a PMC composition described herein is used to treat or prevent cancer or infection with or clinical signs or symptoms caused by a virus.

In another aspect, the disclosure provides a method for administering a nanoparticle as described herein to a subject in need thereof. In some forms, the administration targets a desired body part or organ. In some forms, the organ is selected from the group consisting of liver, spleen, kidney and lung with heart and brain. In some forms, the administration is systemic. In some forms, the administration is via any conventional route including injection and/or intravenously. In some forms, the administration occurs 1, 2, 3, 4, 5, 6, 7, 8, 9, 10, or more times including on a routine basis hourly, daily, bi-daily, weekly, monthly, yearly, or the like. In some forms, the composition administered includes nanoscale physiometacomposite (PMC) materials containing zinc oxide doped with manganese, iron, nickel or cobalt.

Interestingly, while the anticancer activity of ZnO had been reported, Ni/ZnO PMC inhibits melanoma cell invasion and ERK and AKT expression, two markers often associated with drug-resistant cancers.

The Ni/ZnO anticancer activity is enhanced with LL37 peptide, and the ZnO in conjunction with RAS/RBD targeted antisense oligomer or aptamer.

The following examples demonstrate certain aspects of the present disclosure. However, it is to be understood that these examples are for illustration only and do not purport to be wholly definitive as to conditions and scope of this disclosure. It should be appreciated that when typical reaction conditions (e.g., temperature, reaction times, etc.) have been given, the conditions both above and below the specified ranges can also be used, though generally less conveniently. The examples are conducted at room temperature (about 23° C. to about 28° C.) and at atmospheric pressure unless otherwise noted. All parts and percents referred to herein are on a weight basis and all temperatures are expressed in degrees centigrade unless otherwise specified. Further unless noted otherwise, all components of the disclosure are understood to be disclosed to cover “comprising”, “consisting essentially of”, and “consisting of” claim language as those terms are commonly used in patent claims.

### BRIEF DESCRIPTION OF THE DRAWINGS

FIG. 1A is a graph illustrating the fluorescence quenching of ZnO NP wherein at about 525-530 nm, the line that is lowest is RBD, the next lowest line is RBD+0.01 mgmL ZnO, the next lowest line is 0.01 mgmL ZnO, the next lowest line is RBD+0.05 mgmL ZnO, the next lowest line is RBD+0.01 mgmL ZnO, the next lowest line is 0.05 mgmL ZnO, the next lowest line is RBD+0.25 mgmL ZnO, the next lowest line is 0.25 mgmL ZnO, and the highest line is 0.1 mgmL ZnO;

FIG. 1B is a photograph illustrating the RAS binding domain and the ZnO NP;

FIG. 1C is a graph and photograph illustrating the cytotoxicity of the ZnO NPs wherein the lowest line at 20 μg/ml is ZnO-14 and the higher line is ZnO-100;

FIG. 1D is a schematic illustration of the interaction of Ras Binding Domain (RBD) by chemotherapeutic zinc oxide nanoparticles;

FIG. 2 is an illustration of the tail injection used in the experiments where the mouse were imaged live in the bio-imager and also ex vivo to assess tissue histopathology and hematology;

FIG. 3A is an illustration of the synthesis of Cy5.5-ZnO wherein the 3-Mercaptopropionic acid is on the nanoparticle and the MAL-PEG-SCM is attached to the 3-Mercaptopropionic acid;

FIG. 3B is an illustration comparing the fluorescence of Cy5.5 with Cy5.5-ZnO;

FIG. 4A is a series of photographs illustrating the results of the bioimaging of mouse tissues;

FIG. 4B is a series of photographs illustrating the histopathology in multiple tissue types of PBS control (panel 1), ZnO NP (panel 2), and ZnO NP-Cy5.5 wherein each panel the tissues are brain (top left), heart (top middle), lung (top right), spleen (bottom left), liver (bottom middle), and kidney (bottom right);

FIG. 4C is a graph and photograph illustrating the tissue distribution of cy5.5-ZnO/ZnO with the background subtracted wherein the fluorescence is the first portion of each graph line and the amount of zinc is the second portion of each graph line;

FIG. 4D is a summary of the results for the test parameters;

FIG. 5A is a schematic illustration of the process for using zinc-based physiological metal composites (PMCs) to deliver biological activity;

FIG. 5B is a schematic illustration of the PMC conjugates that incorporate RNA;

FIG. 5C is a series of photographs illustrating NP in cells using optical and hyperspectral imaging and ASO and ASO+NP delivery;

FIG. 6 is a series of graphs comparing the DSC analysis for the melting temperature increase for poly I:C upon interaction to ZnO shown by DSC;

FIG. 7 is a graph illustrating the structural stability imparted to poly I:C upon binding to ZnO NP as shown by circular dichroism wherein the peak of the lines occurring between 270 and 290 nm represents Poly(I:C) for the lowest line, Poly(I:C)-ZnO at both 1:1 and 10:1 for the middle line, and Poly(I:C) at 20:1 for the top line;

FIG. 8 is a 2D graph illustrating the fluorescence shift of NP-protein:RNA tripartite complexes formed with Zn-based nanomaterials;

FIG. 9 is a photograph of a RNA agarose electrophoresis (RAGE) illustrating that protamine coating MSN or ZnO NP imparts RNA stability to TY-RNA when incubated at 4° as a PBS suspension;

FIG. 10 is a photograph of a RAGE illustrating the stability of dry powders incubated for 1 or 2 days at 30, 40, or 50° C. in comparison with PBS suspensions stored in the refrigerator for 1 day or 1 week and left out on the bench overnight prior to RAGE analysis;

FIG. 11 is a set of graphs illustrating mRNA expression and delivery activity including in vitro translation of ZnO-protamine-mRNA in the left panel and mean cell fluorescence for delivery of ZnO-P-GFP, ZnO-P-RNA, or ZnO-P-RNA-P in the right panel wherein the top line of the fluorescence is ZnO-P-RNA-P, the middle line of the fluorescence is ZnO-P-RNA, and the bottom line of the fluorescence is ZnO-P-GFP;

FIG. 12 is a graph illustrating the zeta potential analysis of ZnO NP surface charge after coating with protamine at low (L), medium (M), or high (H) concentrations followed by TY-RNA complexation;

FIG. 13 is a photograph of a RAGE analysis illustrating NP protection from degradation in water after overnight incubation at 37° C. wherein MSN=mesoporous silica nanoparticle, Cu=copper nanoparticle, CNT=carbon nanotube, Fe₃O₄=iron oxide nanoparticle, control RNA (TY-RNA), and ZnO=zinc oxide nanoparticle;

FIG. 14 is a graph illustrating RNA loading efficiency by showing the RNA amount remaining that is not particle-associated as a function of coating ZnO NP with cationic peptide protamine or LL-37 (SEQ ID NO. 2);

FIG. 15 is a series of panels illustrating RNA temperature stabilization wherein the top left panel is a graph illustrating that ZnO NP interaction to poly I:C increases melting temperature as shown by DSC, the top right panel illustrates that ZnO NP interaction to poly I:C imparts structural stability to RNA as shown by circular dichroism, wherein the peak of the lines occurring between 270 and 290 nm represents Poly(I:C) for the lowest line, Poly(I:C)-ZnO at both 1:1 and 10:1 for the middle line, and Poly(I:C) at 20:1 for the top line, and the bottom panel illustrates the accelerated stability of ZnO-protamine-RNA liquid formulations;

FIG. 16A is a graph illustrating the hydrodynamic size of ZnO and ZnO-PEG wherein the top line is ZnO and the bottom line is ZnO-PEG;

FIG. 16B is a graph illustrating the zeta potential of ZnO and ZnO-PEG wherein the top line is ZnO-PEG and the bottom line is ZnO;

FIG. 16C is a graph illustrating that ZnO NP fluorescence intensity was stable after incubation in serum for one week;

FIG. 17A is a photograph illustrating the uptake of cy5.5-labeled ZnO NP into Caco-2 3-D organoids;

FIG. 17B is a photograph illustrating cy5.5-ASO delivery into human A375 melanoma cells by ZnO NP or Co3O4 NP relative to cy5.5-ASO control;

FIG. 17C is a graph illustrating the improved uptake by ZnO, Co3O4 or NiO NP confirmed by flow cytometry;

FIG. 17D is a graph illustrating splicing correction of the aberrant RBD transcript by nanoparticle delivery of ASO targeting that cryptic site;

FIG. 18A is an illustration of the PMC nanoparticle derivations;

FIG. 18B is a table illustrating the composition of each PMC nanoparticle that was tested;

FIG. 18C is a graph illustrating the biocompatibility of the different PMC compositions after 48 hours of treatment of continuous exposure in serum containing media to NIH3T3 cells;

FIG. 19A is a photograph illustrating ZnO NP, CoZnO or NiZnO increase cy5.5-poly I:C labeling of 3-D tumor spheroid as shown by bio-imager relative to RNA alone or untreated controls;

FIG. 19B is a schematic and photograph illustrating that NiZnO or higher order PMC containing iron and manganese inhibited 3-D tumor spheroid growth and ablated these structures;

FIG. 19C is a photograph illustrating that ZnO or CoZnO PMC inhibited B16F10 cell invasion in the scratch assay, the PMC treated material still having the gap filled in by untreated or poly I:C negative controls;

FIG. 19D is a graph illustrating the percent cytotoxicity on NiZnO treatment to M5 canine mucosal cells;

FIG. 20A is an illustration of a high throughput proteomic analysis of B16F10/BALB-c tumor;

FIG. 20B is a graph illustrating RBD protein interference;

FIG. 20C is a table showing RBD or BCL-xL targeted ASO in B16F10, A375 or 132N1 cells using SEQ ID NOS 4 and 5, respectively;

FIG. 20D is a graph illustrating relative gene expression in response to Ni/ZnO PMC NP for ERK/AKT RT-PCR;

FIG. 20E is a graph illustrating anti-melanoma activity after treating drug-resistant canine mucosal melanoma cells with LL37 peptide wherein, from left to right, the NiZnO is represented by the 1^{st} and 5^{th} bars, NiZnO-LL37 is represented by the 2^{nd} and 6^{th} bars, ZnO is represented by the 3^{rd} and 7^{th} bars, and ZnO-LL37 is represented by the 4^{th} and the 8^{th} bars;

FIG. 20F is a graph illustrating the effects after treating drug-resistant canine mucosal melanoma cells with Aptamer vs ASO or RBD ASO and aptamer complexes;

FIG. 21A is a graph illustrating the enzyme inhibition at 20 ug/ml PMC via the inhibition of B-Gal biochemical activity by various PMC including ZnO NP or silver nanoparticle control;

FIG. 21B is a genome-wide RNA profile and differential analysis of NSARS-CoV-2;

FIG. 22 is a photograph illustrating the temperature-stability imparted to TY-RNA, when bound to ZnO NP via protamine, dried to a powder and resuspended in sterile PBS can be stored at elevated temperatures for prolonged periods of time (2 weeks); and

FIG. 23 is a graph illustrating the binding of LL37 peptide to various PMC NP compositions as a function of zeta potential surface charge shift.

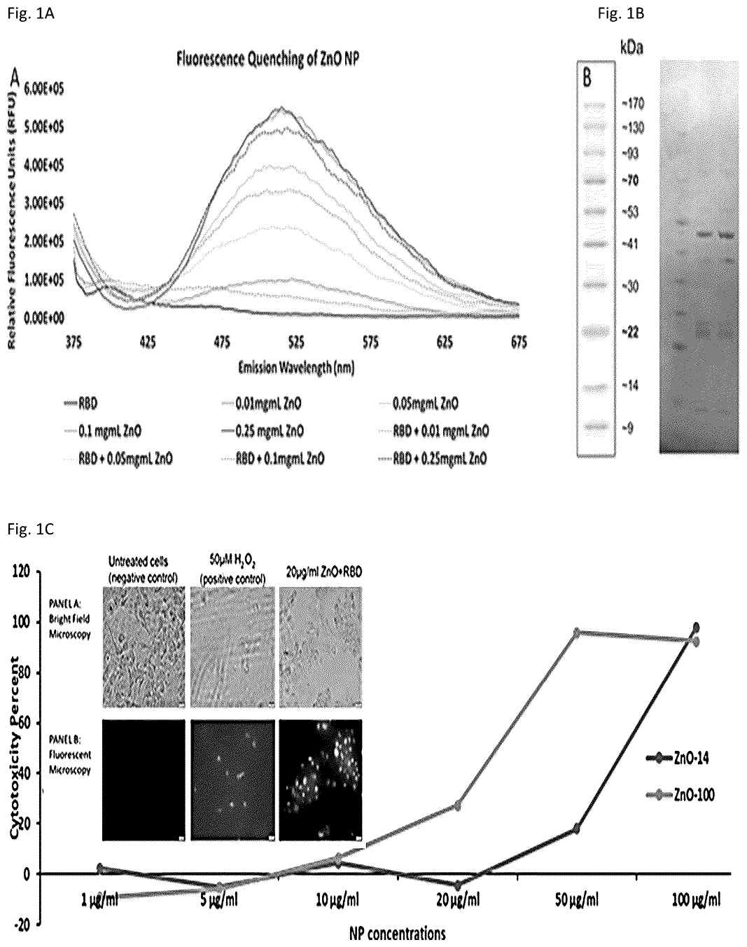

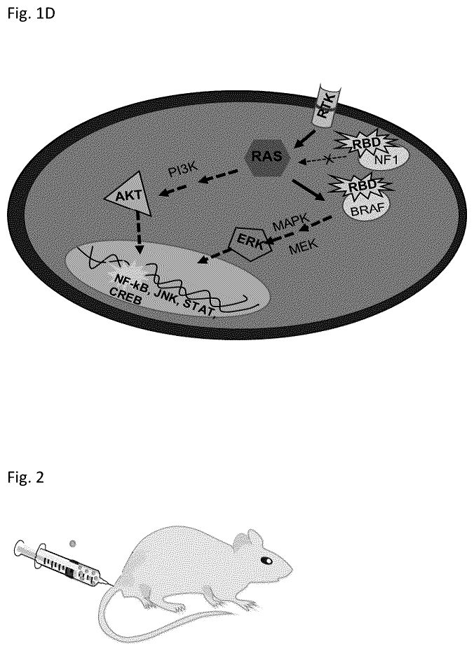

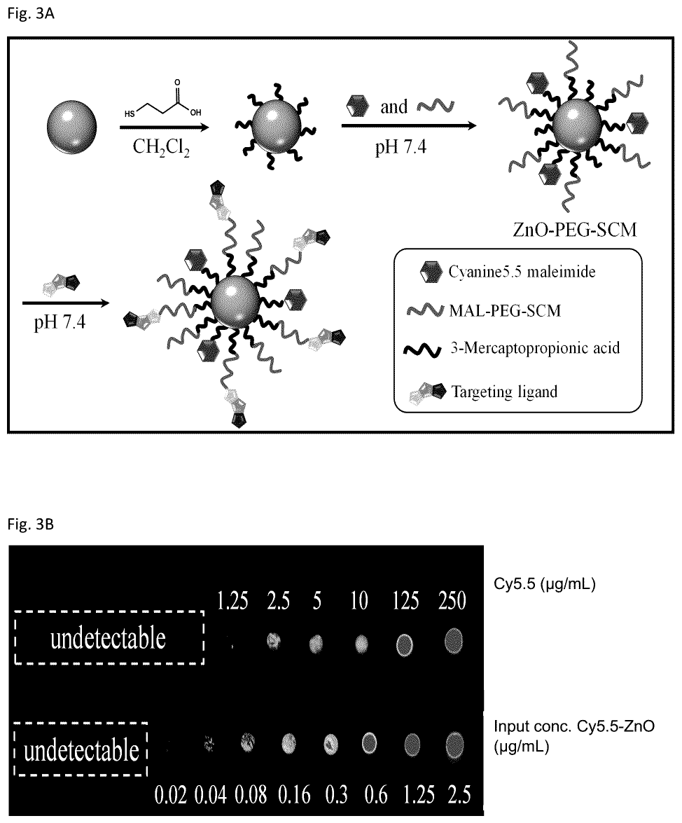

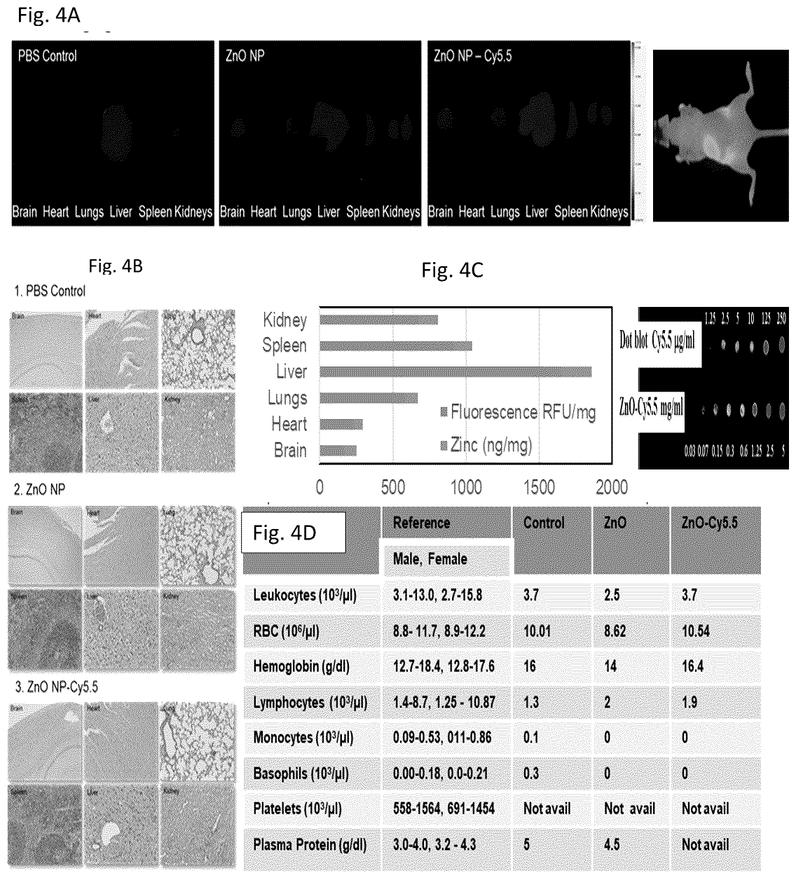

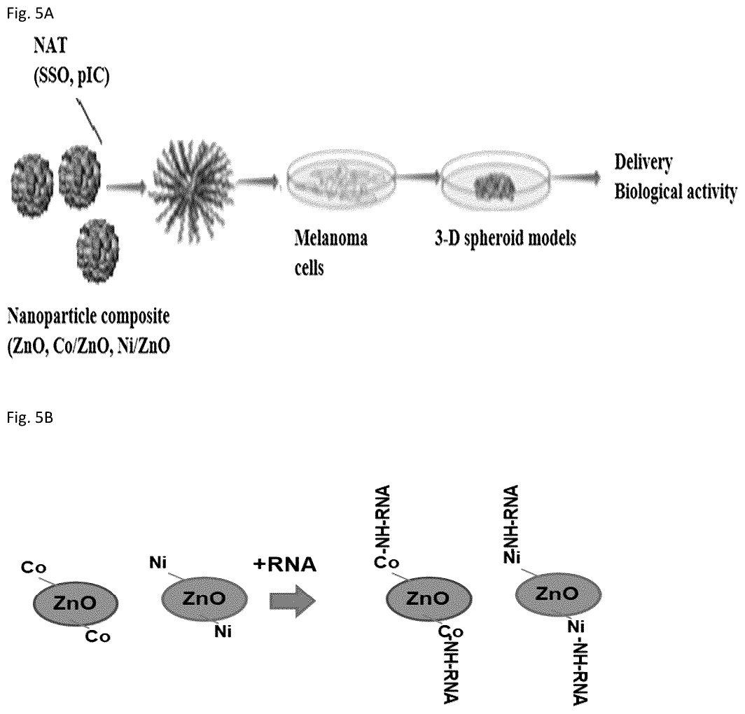

### DETAILED DESCRIPTION OF THE DISCLOSURE

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

### Example 1

### Materials

ZnO: Sigma Aldrich 544906-10G nanopowder<100 nm particle size (PlasmaChem 14 nm has also been used)
Protamine Sulfate: Sigma Aldrich P3369-10G
70% EtOH
100% EtOH
Milli-Q water
SmartGlow Loading Dye, Nucleic Acid Electrophoresis Concentration 6×-Bullseye BE4500-LD 1 mL

Safety and handling considerations: When working with ZnO NP and RNA gloves and a lab coat should be worn at all times and the work should be conducted in a clean bench area so as to minimize contamination with RNases or nucleases which can more generally degrade RNA. When weighing out ZnO NP take care to do so carefully within the controlled environment of the analytical balance to avoid any residual vapor inhalation. Since RNase-free water typically uses chemical treatment, instead use pristine double-distilled or milliQ water which has only ever come in contact with RNase free tips and tubes.

### Prepare Samples

1. If making from scratch:
   a. Weigh out 1.5 mg of ZnO (3 tubes)
   b. Weigh out 1.5 mg of Protamine (1 tube)
2. Wash ZnO
   a. 70% isopropyl alcohol (need 10× water so 1 ml), cap the tubes and finger tap mix to wash the particles and briefly pellet in the microcentrifuge
   b. Carefully take off supernatant, keep pellet “aspirate the supernatant” leaving pellet at the bottom
3. Initially a saturated solution of protamine dissolved in mEq water was used and 33 microliters of a 1:100 dilution in water was used to coat the nanoparticles with the cationic peptide (subsequent experiments are focused on optimizing the protamine concentration, too much and the RNA won't come back off the particle, too little and no stability-enhancement will be conferred).
   a. After adding 33 or 50 microlters of the protamine solution, the eppendorfs are finger tap mixed to coat the particles and the tubes are balanced and spun down to pellet the NPs.
   b. Carefully take off supernatant, keep pellet “aspirate the supernatant” leaving pellet (protamine coated NP) at the bottom
4. Next add the RNA solution. The initial goal was to make 1 ml formulations essentially enough for ten 100 microliter animal vaccination injections where each injection contained 30 mcg per 100 uL)
   a. Therefore a total of 300 micrograms of RNA must be added to each 1.5 ml capacity Eppendorf however because RNA precipitation requires at least 70% ethyl alcohol (ETOH)/water or 70% ispopropanol/water the volume of RNA in the tube should be less than 100 microliters or at most 200 microliters.
   b. Once the appropriate volume of RNA is added depending on the concentration of your stock QS this up to 100 or 200 ul max with water, finger mix a couple of times so that all the RNA in solution comes into contact with the protamine coated NP (this is the binding or complexation step.
   c. Then add 1 ml of ice cold 100% ETOH or IPr-OH and out the tubes. The precipitation is immediate but if time you can let the tubes sit in the −20 deg C. freezer for 20-30 mins
   d. Afterwards pellet the samples now containing the NP-Protamine-RNA “NPRs” (candy coated peanut M&M) at 5,000 RPM for 5 mins
   e. Carefully remove the supernatant you should see a white pellet at the bottom tilted to one side which is your NPRs.
   f. Finger tap mix and roll the side so as to get all the solvent off the side (be sure to remove as much of the liquid as you can leaving only the white pellet at the bottom).
   g. Dry the samples in the sterile hood if destined for cell culture or animal experiments (if in vitro assay only can keep in the chemical hood)

Preliminary studies based on gel analysis suggest the dried particles may be reconstituted in 1 ml of sterile PBS and stored in the refrigerator for several days. Dry powder storage in the refrigerator versus room temperature experiments are currently in progress.

### Eluting the RNA Off the Nanoparticles

Protamine has multiple tracts of arginine (Arg) making it a highly cationic protein which has an avid affinity for binding and condensing DNA and RNA as we and many others have shown. In order to remove the RNA from its association with the protamine coated nanoparticle therefore the elution buffer must contain a highly anionic polymer (we have used heparin in the past) as well as a high salt buffer near neutral pH (PBS/TAE) and a detergent to denature the protein (0.1% SDS). In the initial elution buffer I made we had a brown vial of residual heparin into which I placed 1×PBS/TAE/0.1% SDS and added a small volume of saturated poly acrylic acid so as not to affect the pH too much [please note: we have reported heparin substituted buffers can elute RNA from both dendrimer and inorganic nanoparticle complexes although that was a minor aspect of those earlier studies. With the great importance now of all different types of RNA nanoparticles developing a buffer which can remove RNA from different nanoparticles yet maintain the integrity of the RNA molecule is very important].
a. To test the stability of the RNA at different time-points and temperatures to the 1 mg NPRs samples can be added one ml of PBS to re-suspend the sample, and then each 100 microliter sample can be precipitated with 100% alcohol as previous
b. A small white pellet at the very bottom of the tube will be noticeable in which case the supernatant is removed and 20-30 microliters of elution buffer is added, the tubes are finger mixed and incubated at 37 deg C. for 20-30 mins (this removes the RNA away from the nanoparticle and protamine)
c. After which the entire aliquot is loaded onto the gel for analysis (or could be loaded into the CD or DCS for structural or melting temperature analysis)

### Making Agar Gel

Always wear gloves when working with RNA

Make your own gel-RNA specific

Parafilm on the outside to block the gel from running off the platform.

Agar: GP2
1. Weigh out: 2% gel 2 g to 100 mL
2. Add TAE Buffer: 98 mLs 0.5×TAE=1(50×):100
   a. when dissolving agar, should be charge across the gel need salt and buffer in the gel to transfer the charge
3. Heat in microwave—
   a. Heat 20 sec, then 2×10 sec bursts, swirl in between each heating, may have to 3×5 sec bursts
   b. Cool off gel (until you're able to touch the glass) for about 30 sec
   c. Set up the gel platform by using parafilm to seal each side. Add a little solution buffer and swish around to make sure there aren't any leaks before adding Agar to the gel platform
   d. Place in the comb. Then add agarose gel.
   e. Let sit for 20-30 min
4. Check the Gel
   a. Make sure the wells are set by moving the comb from side to side. If it doesn't seem like the gel is clinging on to the comb, slowly ease it out of the gel. Move them around without ripping them out. Might leave ridges if not properly set
5. Set up the gel “box”
   a. Wells on left side.
   b. RNa=negative, positive (red) charge added on right side “Run to Red”
   c. Negative (black) charge plugged in on left side
6. Add running buffer (50×:100 diluted solution-either 1× or 0.5×TAE)—enough so that it covers the top

### How to Load Each Well

Take parafilm, and create dots—as much as there are samples
1. Give each dot 1 microL of staining dye+saturated sucrose
   a. To allow for RNA gel to get in, need to add saturated sucrose—adds weight and goes down to the well to prevent escape into “ether of space”
   2. Add 10 microL of saturated sucrose (can wash out pipette before adding to next sample)
   3. Control: TyRNA 1 microL or 0.5 microL add to gel
   4. Load the prepared sample into the well by pulling up 20 microL (total volume) off the parafilm

### Running the Gel

Load the Wells

Turn on Power button, Manual 100 V, hit run, let run for 1 hr (at least)

Setting up times for Gel Imaging
Equipments in nanolab: Bio Rad Gel Doc XR
Run the gel about an hour
Give experiment a name:
   example— Initials, RNA, time and temp stability
   Save the gel image with a date and back it up on a flashdrive

### Example 2

### Materials

ZnO:
a. Sigma Aldrich 544906-10G nanopowder<100 nm particle size
b. PlasmaChem 14 nm
Protamine Sulfate: Sigma Aldrich P3369-10G
Ribonucleic acid from torula yeast; Type VI R6625-25G Sigma
Aldrich
70% EtOH
100% EtOH
Milli-Q water
SmartGlow Loading Dye, Nucleic Acid Electrophoresis
Concentration 6×-Bullseye BE4500-LD 1 mL

### Safety and Handling Considerations:

When working with ZnO NP and RNA gloves and a lab coat should be worn at all times and the work should be conducted in a clean bench area so as to minimize contamination with RNases or nucleases which can more generally degrade RNA. When weighing out ZnO NP take care to do so carefully within the controlled environment of the analytical balance to avoid any residual vapor inhalation. Since RNase-free water typically uses chemical treatment, instead use pristine double-distilled or milliQ water which has only ever come in contact with RNase free tips and tubes.

### Prepare Samples

1. If making from scratch:
2. Weigh out 1.5 mg of ZnO (3 tubes)
3. Weigh out 1.5 mg of Protamine (1 tube)+2 empty tubes
4. Weigh out 3 mg of TY RNA (1 tube)
5. Wash ZnO
6. 70% isopropyl alcohol (need 10× water so 1 ml), cap the tubes and finger tap mix to wash the particles and briefly pellet in the microcentrifuge at 5000 RPM for 5 min.
7. Carefully aspirate the supernatant, leaving white pellet at the bottom
8. If there is still isopropyl alcohol still left over, spin again and aspirate the supernatant that's left for a cleaner, drier sample.

Initially a saturated solution of protamine dissolved in milli-q water (1 mL total) was used and 33 microliters of a 1:100 dilution in water was used to coat the nanoparticles with the cationic peptide. Subsequent experiments are focused on optimizing the protamine concentration, too much and the RNA won't come back off the particle, too little and no stability-enhancement will be conferred.
1. 1 mL of milli-q water is added to 1.5 mg of Protamine in the Eppendorf.
2. Serial Dilution method was used to create Protamine at different concentrations. Dilutions were created by taking 1/10^{th} (100 uL) from the previous Eppendorf.
   a. High: 1:1
   b. Medium: 1:10
   c. Low: 1:100
a. From each of these concentrations of protamine, 33 uL were taken from the protamine solution to the 3 washed ZnO Eppendorf. Label the ZnO Eppendorf according to dilution ratio. They are finger tap mixed to coat the particles.
b. Next, the tubes are balanced and spun down to pellet the NPs at 5000 rpm for 5 min.
c. Aspirate the supernatant leaving pellet (protamine coated NP) at the bottom. Spin again if needed.
1. Create RNA Stock Solution:
   a. Take Eppendorf that contains 3 mg of TY-RNA and dissolve in 1 mL milli-q water.
2. Next add the RNA solution to each ZnO+Protamine Eppendorf. The initial goal was to make 1 ml formulations essentially enough for ten 100 microliter animal vaccination injections where each injection contained 30 mcg per 100 uL)
   a. Therefore a total of 30 mcg of RNA must be added to each 1.5 ml capacity Eppendorf. However, because RNA precipitation requires at least 70% ethyl alcohol (ETOH)/water or 70% ispopropanol/water the volume of RNA in the tube should be less than 100 microliters or at most 200 microliters.
   b. Add 100 uL of RNA Stock Solution to each ZnO+Protamine Eppendorf. Finger mix a couple of times so that all the RNA in solution comes into contact with the protamine coated NP. (This is the binding or complexation step.)
3. Now wash the ZnO+Protamine+RNA with ETOH or IPr-OH to precipitate the sample.
   a. Add 1 ml of ice cold 100% ETOH or IPr-OH and out the tubes. The precipitation is immediate but if time you can let the tubes sit in the −20 deg C. freezer for 20-30 mins
   b. Afterwards pellet the samples now containing the NP-Protamine-RNA “NPRs” at 5,000 RPM for 5 mins c. Carefully remove the supernatant you should see a white pellet at the bottom tilted to one side which is your NPRs.
   d. Finger tap, mix and roll the side so as to get all the solvent off the side (be sure to remove as much of the liquid as you can leaving only the white pellet at the bottom).
   e. Dry the samples (24 hours) in the sterile hood if destined for cell culture or animal experiments (if in vitro assay only can keep in the chemical hood)

Preliminary studies based on gel analysis suggest the dried particles may be reconstituted in 1 ml of sterile PBS and stored in the refrigerator for several days. Dry powder storage in the refrigerator versus room temperature experiments are currently in progress.

### Eluting the RNA Off the Nanoparticles

Protamine has multiple tracts of arginine (Arg) making it a highly cationic protein which has an avid affinity for binding and condensing DNA and RNA. In order to remove the RNA from its association with the protamine coated nanoparticle therefore the elution buffer must contain a highly anionic polymer (we have used heparin in the past) as well as a high salt buffer near neutral pH (PBS/TAE) and a detergent to denature the protein (0.1% SDS). In the initial elution buffer I made we had a brown vial of residual heparin into which I placed 1×PBS/TAE/0.1% SDS and added a small volume of saturated poly acrylic acid so as not to affect the pH too much.

Re-suspend the ZnO+Protamine+RNA in 1 mL of PBS.
1. To test the stability of the RNA at different time-points and temperatures to the 1 mg NPRs samples can be added one ml of PBS to re-suspend the sample.
2. Take out one “dose” worth of the sample and precipitate with alcohol.
3. Take out 100 microlter sample from PBS suspended sample and precipitate with cold 100% ETOH alcohol as previous.
4. A small white pellet at the very bottom of the tube will be noticeable in which case the supernatant is removed and 20-30 microlters of elution buffer is added, the tubes are finger mixed and incubated at 37 deg C. for
20-30 mins (this removes the RNA away from the nanoparticle and protamine)
5. After which the entire aliquot is loaded onto the gel for analysis (or could be loaded into the CD or DCS for structural or melting temperature analysis). To load the sample onto the gel, more steps must be taken for proper visualization.

### Making Agar Gel

Always wear gloves when working with RNA

Make your own gel-RNA specific
Before creating agar gel, place parafilm on the outside edges to block the gel from running off the platform. Place comb to allow for well creation.
Check seal of parafilm by placing a few drops of TAE buffer and moving it along the edges. If not properly sealed, redo the parafilm and check again.

Agar: GP2
i. Weigh out: 2% gel 2 g to 100 mL
ii. Add TAE Buffer: 98 mLs 0.5×TAE=1(50×):100; 2 mL of 50×TAE to 98 mL DI water
iii. when dissolving agar, should be charge across the gel need salt and buffer in the gel to transfer the charge
iv. Heat in microwave—
v. Heat 20 sec, then 2×10 sec bursts, swirl in between each heating, may have to 3×5 sec bursts
vi. Cool off gel (until you're able to touch the glass) for about 30 sec
vii. Set up the gel platform by using parafilm to seal each side. Add a little solution buffer and swish around to make sure there aren't any leaks before adding Agar to the gel platform
viii. Place in the comb. Then add agarose gel.
ix. Let sit for 20-30 min
x. Check the Gel
   1. Make sure the wells are set by moving the comb from side to side. If it doesn't seem like the gel is clinging on to the comb, slowly ease it out of the gel. Move them around without ripping them out. Might leave ridges if not properly set.
xi. Set up the gel “box”
   1. Wells on left side.
   2. RNa=negative, positive (red) charge added on right side “Run to Red”
   3. Negative (black) charge plugged in on left side xii. Add running buffer (50×:100 diluted solution-either
1× or 0.5×TAE)—enough so that it covers the top

### How to Load Each Well

Take parafilm, and create dots—as much as there are samples+control
a. Give each dot 1 microL of bulls eye staining dye+10 microL of saturated sucrose
b. To allow for RNA gel to get in, need to add saturated sucrose—adds weight and goes down to the well to prevent escape into “ether of space”
c. Add 10 microL of saturated sucrose (can wash out pipette before adding to next sample)
d. Control: TyRNA 1 microL or 0.5 microL add to gel
e. Load the prepared sample into the well by pulling up 20-30 microL (total volume) off the parafilm

RUNNING THE GEL

Load the Wells

Turn on Power button, Manual 100 V, hit run, let run for 1 hr (at least)

Setting up times for Gel Imaging
Equipments in nanolab: Bio Rad Gel Doc XR
Run the gel about an hour
Give experiment a name:
   example—Initials, RNA, time and temp stability
Save the gel image with a date and back it up on a flashdrive

### Example 3

### Materials and Methods

Differential scanning calorimetry (DSC) was conducted by Dr. Akshay Jain at the University of Kansas Center for Vaccine Stabilization and Characterization according to a protocol developed by the group for protein vaccine subunits here applied to poly I:C exposed to 20 ug/ml ZnO NP in phosphate buffered saline. Circular dichroism experiments were conducted as we have previously described. Poly inosinic:poly cytidylic acid [poly(I:C)] and Torula yeast RNA (TY-RNA) were obtained from Sigma-Aldrich. 100 nm and 14 nm ZnO NP were obtained from Sigma-Aldrich and PlasmaChem respectively. MgO NP was obtained from Sigma-Aldrich. Cobalt oxide and nickel oxide nanoparticles were synthesized by Dr. Kartik Ghosh as previously reported. ASO sequence: (3-CCUCUUACCUCAGUUACA-5) (SEQ ID NO. 1) was obtained from Trilink Biotechnologies. Zeta potential measurements and UV payload experiments were conducted as previously described in Comparative functional dynamics studies on the enzyme nano-bio interface. Thomas S E, Comer J, Kim M J, Marroquin S, Murthy V, Ramani M, Hopke T G, McCall J, Choi S O, DeLong R K. Thomas S E, et al. Int J Nanomedicine. 2018 Aug. 8; 13:4523-4536; Comparing the effects of physiologically-based metal oxide nanoparticles on ribonucleic acid and RAS/RBD-targeted triplex and aptamer interactions, Huslig G, Marchell N, Hoffman A, Park S, Choi S O, DeLong R K. Huslig G, et al. Biochem Biophys Res Commun. 2019 Sep. 10; 517(1):43-48; and Amino/Amido Conjugates Form to Nanoscale Cobalt Physiometacomposite (PMC) Materials Functionally Delivering Nucleic Acid Therapeutic to Nucleus Enhancing Anticancer Activity via RAS-targeted Protein Interference. RK DeLong, J Dean, G Glaspell, M Jaberi-Douraki, K Ghosh, D Davis, Nancy Moneteiro-Riviere, P. Chandran, T. Ngyuen, S. Aryal, C. Russel Middaugh, S. Chan Park, S-O Choi and Meghana Ramani. ACS Applied Bio Materials 3 (1), 175-179 2020.

### Results and Discussion

### Zeta Potential and Payload Analysis:

In cells and tissues, zinc is used to stabilize RNA and protein structure and interactions. Thus using zeta potential analysis the change in apparent charge at the surface of the nanoparticle was assessed, comparing RNA interaction to zinc oxide nanoparticle (ZnO NP) versus the other common physiologically-based metal oxide nanoparticles with a known active nanobio interface. These data are summarized in Table 1 below:

| Nanoparticle type | Naked | +poly I:C | +ASO | RNA payload (ug/mg) |
|---|---|---|---|---|
| Zinc oxide (ZnO) | −17.2 +/− 0.91 | −28.3 +/− 0.66 | −21.7 +/− 0.17 | 2.30 +/− 0.250 |
| Cobalt oxide (Co₃O₄) | −13.46 +/− 2.1 | −14.3 +/− 8.4 | −31.8 +/− 0.31 | 1.90 +/− 0.050 |
| Magnesium oxide (MgO) | −7.73 +/− 4.2 | −21.8 +/− 0.71 | −13.3 +/− 0.76 | 2.05 +/− 0.070 |
| Nickel oxide (NiO) | −18.2 +/− 6.2 | −22.8 +/− 6.2 | −3.4 +/− 1.5 | 2.00 +/− 0.040 |

Table 1 shows RNA interaction to the physiologically-based metal oxide nanoparticles on the basis of apparent charge at the nanoparticle surface indicated by zeta potential (ZP) analysis, where notably all nanoparticles undergo an anionic shift to the negative in the presence of either antisense oligomer (ASO) or poly I:C. Interestingly the change in ZP for the larger RNA was greatest for ZnO NP consistent with our prior work, whereas ASO interaction favored Co3O4, consistent with its bioconjugation. The RNA payload in units of micrograms/milligram nanoparticle was then obtained by microcentrifugation of the RNA and nanoparticle sample, the loss of UV absorbance in the supernatant when the RNA and nanoparticle controls were background subtracted was used to estimate the payload of RNA per nanoparticle mass. This parameter was significant with the payload increasing dramatically.

### Zinc Oxide Nanoparticle Increases RNA Melting Temperature:

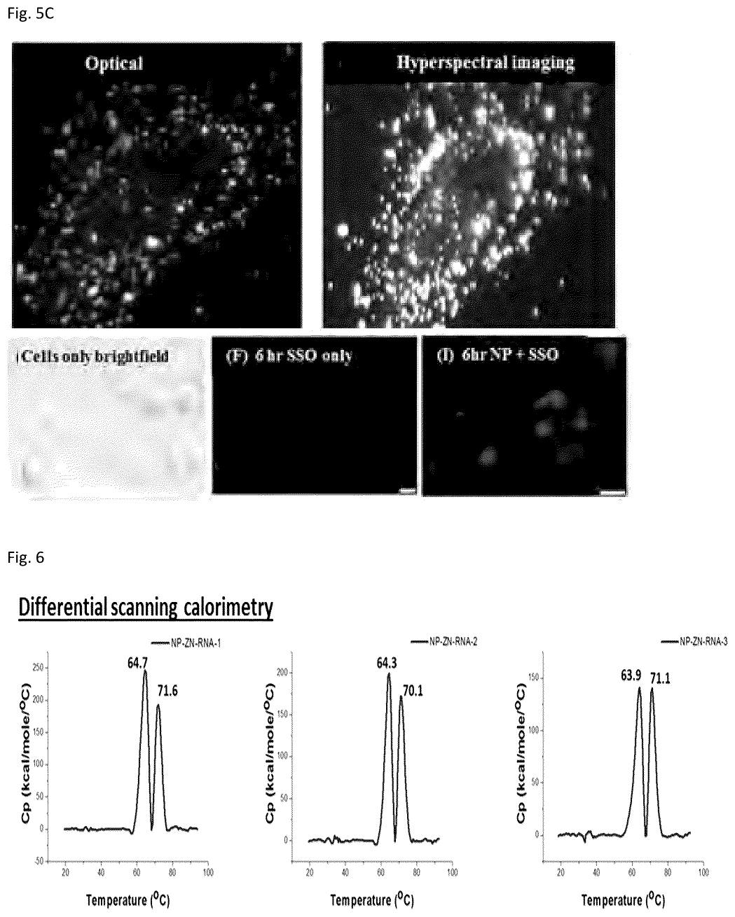

We had previously examined the effect of the physiological metal oxides on duplex and triplex formation and on RNA secondary structure analysis for a microbial RNA obtainable in bulk, torula yeast RNA (TY-RNA) by UV or circular dichroism analysis. Poly I:C is an immunologically active double-stranded RNA viral mimetic and in the presence of ZnO NP its melting temperature shifts from 63.9-64.7 degrees Celsius (° C.) to 70.1-71.6 indicating gain in temperature-stability (FIG. 6):

### Zinc Oxide Nanoparticle Binding Imparts RNA Structural Stability:

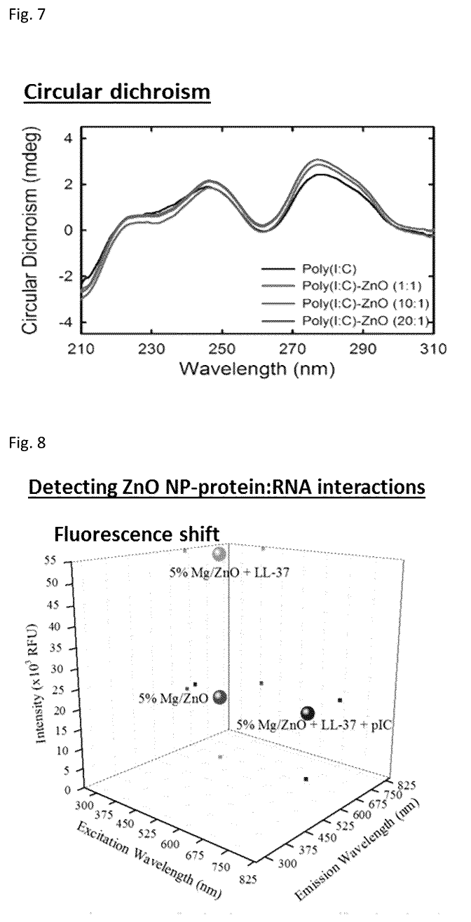

Previously we had examined the effect of physiological nanoparticle interaction to TY-RNA by circular dichroism. Given the temperature-stabilization shown above, we examined the effect of increasing the stoichiometric ratio of ZnO NP to poly I:C by CD similarly. Surprisingly, the presence of ZnO NP at lower stoichiometries, 1:10 or 1:20 actually stabilized the RNA causing the pattern to amplify suggesting structural stability was imparted to the RNA by its interaction to ZnO NP (FIG. 7):

As shown in FIG. 7, the CD pattern is actually accentuated in the presence of ZnO NP suggesting structural stability is imparted to the RNA upon ZnO NP interaction. These data are consistent with the data above, that ZnO NP interaction to poly I:C is RNA-stabilizing.

### NP-Protein:RNA Tri-Complexes

We had previously reported that 2-dimensional fluorescence difference spectroscopy (2-D FDS) could be used to detect ZnO NP surface modification and interaction to RNA. Mg/ZnO nanomaterials were incubated with antiviral LL-37 peptide (SEQ ID NO. 2) with/without poly I:C (pIC) and the fluorescence shift and gel electrophoresis mobility shift as a function of the NP:protein:RNA tripartite species is shown (FIG. 8).

The 2-D FDS shift that occurs when Mg/ZnO interacts with LL-37 (SEQ ID NO. 2) and subsequent RNA interaction to form the tripartite species is shown in FIG. 8. These data suggest that the surface of zinc-based nanoparticles can be coated with cationic protein enabling the subsequent attachment of the RNA.

Protamine Coated Nanoparticles Protect RNA from Temperature Degradation:

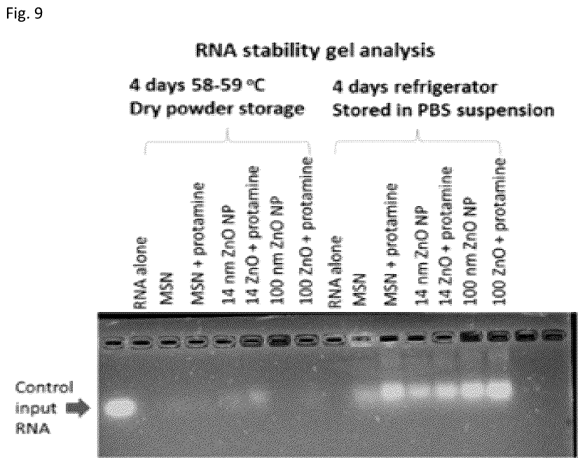

In our previous work we reported protamine could condense DNA or RNA into nanoparticles which could be loaded onto an inorganic surface such as gold and this could impart accelerated stability to DNA vaccine allowing the plasmid DNA vector to retain gel staining intensity. Previously it was reported surface-functionalized mesoporous silica nanoparticle (MSN) could temperature stabilize RNA, and here it could be shown that coating the surface of either MSN or ZnO NP with protamine enhanced the stability of the RNA as shown by RNA agarose gel electrophoresis (RAGE) analysis when incubated at 4 degrees Celsius (4° C.) for up to 4 days when stored as a suspension in PBS buffer (FIG. 9).

As can be seen in FIG. 9, the RNA band staining intensity is retained when the samples, either MSN or ZnO NP are coated with protamine and can be stored in the refrigerator for up to 4 days without losing band intensity. By contrast formulations what were dried to a powder and stored near 60° C. for the same amount of time, very little intact RNA could be detected. It should be noted that in the previous paper, MSN was surface-functionalized prior to RNA loading which protected the RNA and protamine was not used in these experiments. However in that case, RT-PCR amplification was used as a read-out for stability enhancement, and the RAGE method is expected to be a truer reflection of the degree to which RNA structure is maintained over the time course.

### Accelerated Stability Protocol:

TY-RNA was formulated onto ZnO NP (14 nm) by coating first with protamine, alcohol precipitated, air dried and incubated for 1 or 2 days at 30, 40 and 50° C., the RNA eluted from the particles and analyzed by RAGE as shown (FIG. 10).

As can be seen in FIG. 10, when stored as a dry powder, the ZnO-protamine-RNA formulations are stable at 30 or 40 degrees Celsius for several days, the RNA band retaining considerable staining intensity. The formulations stored at 4 deg C. for 1 day or 1 week could also be stored at room temperature as a dry powder and considerable intact RNA could still be detected. These data indicate that the protamine coated ZnO NP can temperature stabilize RNA, both as a suspension in PBS and as a dry powder.

### Example 4 Martials and Methods

Formulation preparation: The samples were made by weighing out 1.5 mg of Zinc oxide nanoparticles (ZnO) (Sigma Alrich 100 nm) or (PlasmaChem 14 nm) and 1.5 mg of protamine (Pr). ZnO was first washed with 1 ml of 70% isopropyl, microcentrifuged at 5000 rpm to 5 minutes, and the supernatant was aspirated. The protamine was prepared by dissolving it in 1 ml of milli-q water (Prot-hi). 3 different dilutions of protamine were created by taking 100 μl of Pr solution and adding to another tube and adding 900 μl of milli-q water to make a dilution of 1:10 (Prot-med). Another dilution of 1:100 was made from taking 100 μl from 1:10 and adding 900 μl of milli-q water (Prot-Lo) Once all 3 dilutions of Pr were created 33 μl were added to the ZnO and mixed. These were microcentrifuged at 5000 rpm for 5 minutes and supernatant was aspirated. The RNA solution was made from 3 mg to 1 ml of milli-q water. 50 to 100 μl RNA (3 mg/ml) was added to the ZnO and Pr sample, mixed, and washed with 1 ml of 100% cold alcohol, ethanol or isopropanol. The sample was microcentrifuged at 5000 rpm at 5 minutes and supernatant was aspirated. The samples were left to dry overnight in their certain temperatures of either 20, 30, 40, or 50° C. To re-suspend the sample, 1 ml of PBS was added to the samples. 100 μl was added into another tube and 1 ml of cold alcohol was added for reprecipitation, then microcentrifuged at 5000 rpm for 5 minutes and supernatant was aspirated. Samples were air dried briefly prior to RNA elution and RAGE analysis.

RNA elution: An initial elution buffer combined saturated heparin and poly-acrylic acid with 10×PBS and 50×TAE and 10% SDS at 1:1:1:1:1 vol:vol. In a scaled up second batch of elution buffer we used crude heparin washed with 70% ETOH/water, dried to a powder the base solution being saturated with this second lot of Heparin buffered again by TAE/PBS with adjustment to 0.1% SDS. In a subsequent single agent screening experiment we noted that the addition of saturated urea in the presence of TAE provided a high intensity RNA band from a formulation prepared as above eluted and analyzed by methods described below.

Stability analyses: Dry powder or PBS re-suspended formulations were maintained in stability chambers (30, 40 and 50° C.) for one to two days, one to two weeks or the formulations or the RNA stock solution stored in the refrigerator for the 4° C. samples. In the hydrolysis experiment, 1 mg/ml nanoparticle was exposed to 1 mg/ml RNA overnight in a hot plate set to 37° C., the samples removed and analyzed by RAGE.

RAGE analysis: To each 100 ul scale dried precipitate aliquot is added 30 μl elution buffer and incubated at 37° C. for 20 minutes. The agar gel was made by weighing out 2% gel 2 g to 100 ml of 1×TAE that was made by 2 ml of 50×TAE and 98 ml distilled water. The solution was heated up in the microwave for 10 seconds and then did 5 second intervals until the solution had become clear. The agarose gel was then cooled off and added to the gel platform with the comb. This was left for 20 minutes to mold into the gel. The samples were prepared for the gel by mixing 30 μl of the samples with 1 μl bullseyes staining and 10 μl saturated sucrose. A total of 30 μl was added into each well. The gel was run at manual 100V for an hour and imaged in the gel imager.

In vitro translation: CleanCap FLuc mRNA was obtained from Trilink Biotechnologies. Nuclease-free Rabbit reticulocyte lysate (RRL) was obtained from Promega. In a typical translation reaction to 1.5 uL of mRNA was added one microliter each of Cys, Leu and Met amino acids in a tube containing biocompatible concentration of NP (20 microgram/mL). After which 35 microliters of RRL was added and the tubes were incubated for 30 minutes at 30 deg C. to allow for translation of the Luc mRNA to protein. Afterwards 100 uL of Luciferin/Luciferase reagent was added and the bioluminescence of the samples were determined at 562 nm Biotek Hybrid Synergy.

Payload efficiency: Tubes were run in triplicate containing RNA control, RNA with protamine or RNA with LL37 (SEQ ID NO. 2). 1 mg of 100 nm ZnO NP was washed with alcohol, to which was added 300 uL of peptide (Prot 1:10) or LL-37 (1:5), spun down and the RNA added (5 uls of 3 mg/ml), spun-down (in the absence of alcohol), and 5 uL aliquots removed and quantified by nano-drop.

Characterization: Particle size and zeta potential analysis were conducted as previously described. CD analysis was conducted as previously described. DSC experiments were conducted in the Vaccine Characterization and Stabilization Center at the University of Kansas in the laboratory of Dr. Russ Middaugh.

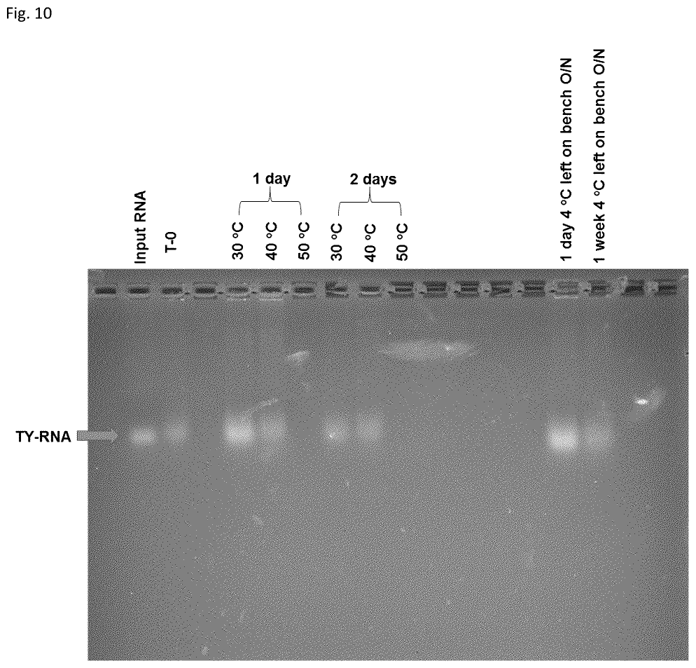

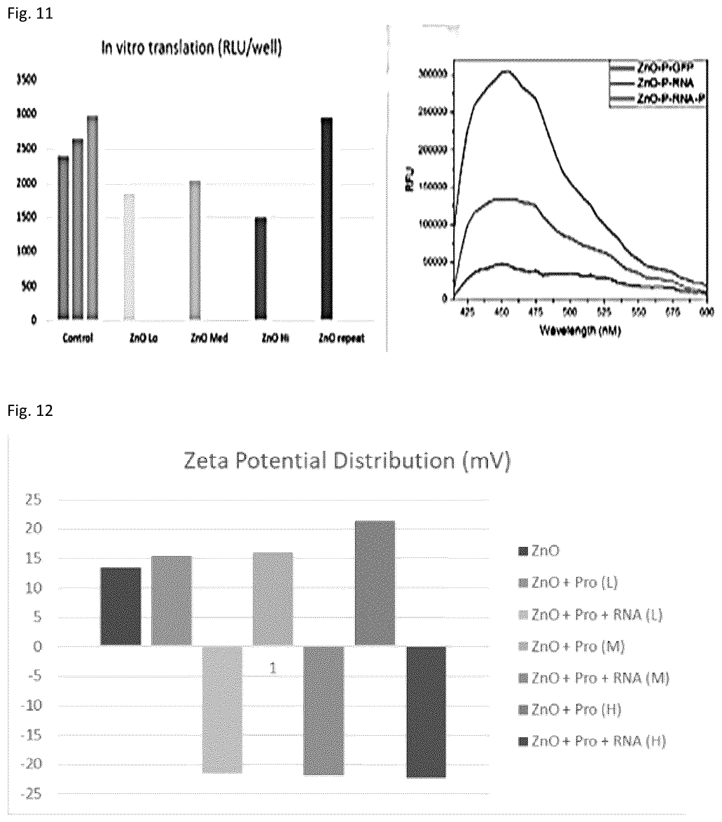

Delivery: NIH 3T3 cells were seeded in 24-well plates at a density of 30,000 cells per square centimeter. 7.4 mg of protamine sulfate were dissolved in 5 milliliters of deionized water autoclaved water. The solution was sonicated for 1 min, and the solution was divided into 1.5 mg of protamine. Each vial was centrifuged, and the supernatant was discarded. The precipitate was re-suspended in 50 mcl of cold 75% ethanol and centrifuged. The alcohol was removed. 50 mcl of DI autoclaved water was added to all vials, and 7 mcl of 1.5 mg/mL protamine solution was added. Each vial was vortexed and centrifuged at 5000 rpm for 5 min. After the removal of the supernatant, 50 mcl of DD water was added to all vials. Then, green fluorescent protein to one vial and green fluorescent protein mRNA was added to the other two vials. 7 mcl of protamine solution was added to one of the vials containing green fluorescent protein mRNA. 100 mcl of cold 75% ethanol were added to all vials to further sterilize the samples; ethanol was removed by centrifugation. Each formulation was suspended in 2 ml of DMEM, all samples dispersed readily into solution. Then, cells were treated with 20 mcg per ml Zinc Oxide formulation for 12 hrs. following seeding. After 24 hrs. Incubation, the treatment was removed and replaced with 10% FBS in DMEM. Cells were allowed to incubate another 24 hrs and the mean cell fluorescence per well analyzed on our Molecular Devices SpectraMax Paradigm instrument. 4 wells were used per condition and the experiments were repeated twice both showing increased mRNA transfection relative to the GFP protein control. The plots shown in FIG. 11 represent the average of all 4 wells for one independent experiment.

Zeta potential analysis demonstrating LBL assembly of ZnO-Protamine-RNA: In our prior plasmid DNA vaccine work, DNA loading and delivery was improved by coating the particles first in USP-grade protamine cationic cell penetrating peptide. Zeta potential measures the effective surface charge of nanoparticle and we reasoned that protamine coating, may similarly create a cationic surface in order to bind more RNA to the nanoparticle. To examine this we incubated ZnO NP with low, medium or high concentrations of protamine and measured the zeta potential before and after protamine coating and complexation to TY-RNA (FIG. 12).

### Results and Discussion

An early report suggested RNA temperature stabilization by surface-functionalized mesoporous silica nanoparticles (MSN) allowing brief storage of RNA at 4° C. In addition to MSN, carbon nanotube (CNT) and several other inorganic nanoparticles have been used for RNA delivery including; silver (Ag), iron oxide (Fe₂O₃/Fe3O4) and copper-composites.

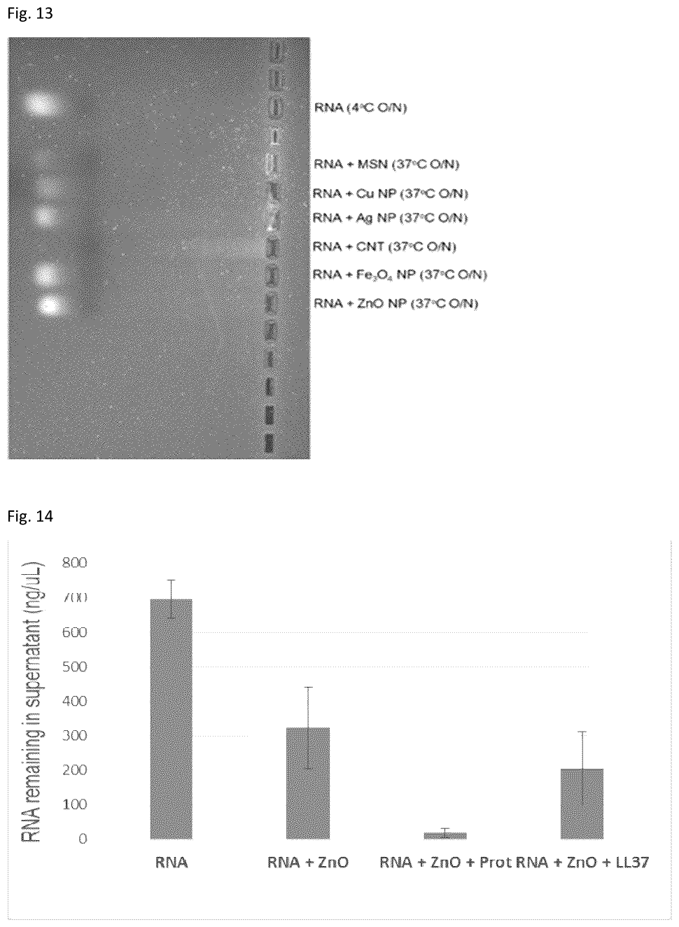

Thus in a first seminal experiment these major types of nanoparticles were compared for their ability to stabilize RNA and protect it from hydrolysis when incubated at physiological temperature (37° C.). FIG. 13

As shown in FIG. 13, RNA stability after incubating torula yeast RNA (TY-RNA) with the nanoparticles in water at 37° C. followed by RNA agarose gel electrophoresis (RAGE) analysis in the following order: ZnO>Fe₃O₄>Ag>MSN Cu>CNT. ZnO NP and to a lesser extent Fe₃O₄, causes a slight gel shift consistent with their RNA interaction, but importantly retain band staining intensity, and the band is tighter in comparison to the control RNA incubated over-night in water at 4° C. indicating some hydrolysis occurs in the control RNA but is protected in the ZnO NP and Fe₃O₄ samples. A demonstrable lack of RNA stain intensity or a smear pattern consistent with RNA degradation was seen with MSN, copper (Cu) NP and CNT. These data are consistent with our previous report which showed copper (Cu) NP denatures RNA.

In our previous clinically approved formulation, protamine was used to coat the surface of gold particles for improved nucleic acid loading. Protamine is considered a nucleic acid condensing agent, and we showed that protamine could condense RNA into nanoparticles active for delivery of RNA into cells. Protamine is a well characterized cationic peptide and there is some work to suggest that it may serve as a cell penetrating peptide with in vivo RNA delivery potential and application for mRNA transfection. Coating ZnO NP with protamine essentially creates a more cationic surface for layer-by-layer (LBL) assembly of RNA onto the surface and this was confirmed by zeta potential analysis, that the anionic surface of the ZnO—NP shifted to cationic when coated with protamine, and then underwent an anionic shift when the layer of RNA was added (FIG. 12). Similar to protamine, LL-37 (SEQ ID NO. 2) (SEQ ID NO. 2) is a peptide with multiple lysine (Lys) and Arginine (Arg) cationic amino acids. LL-37 is thus another cationic peptide of considerable interest recently because it has been shown to have some antiviral activity and a potential role in regulating the immune response against SARS-CoV-2. Next, loading efficiency of RNA onto ZnO—NP coated with Prot or LL-37 was compared (FIG. 14).

As shown in FIG. 14, loss of RNA remaining in supernatant as measured by UV/Vis could be used to estimate loading capacity. Loading capacity is only 40-50% for LL-37, but >95% (<700 ng/uL) for Prot. Thus RNA could be captured quantitatively on ZnO—NP with a single protamine layer coating approach.

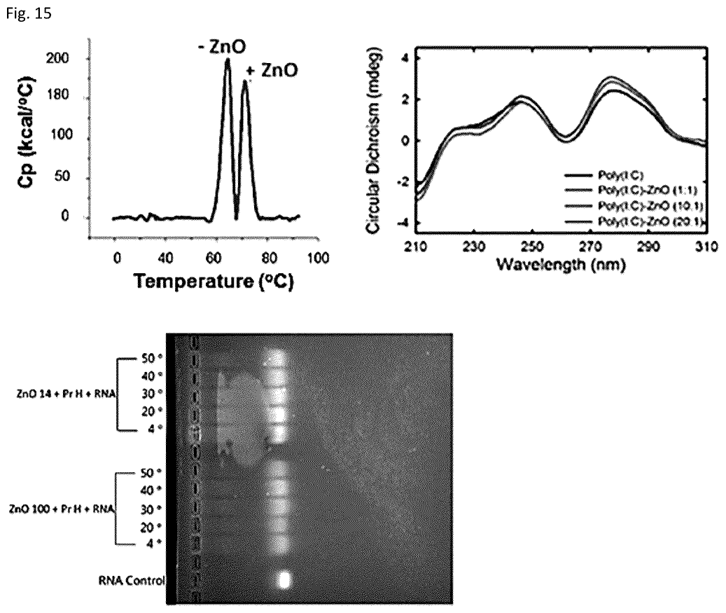

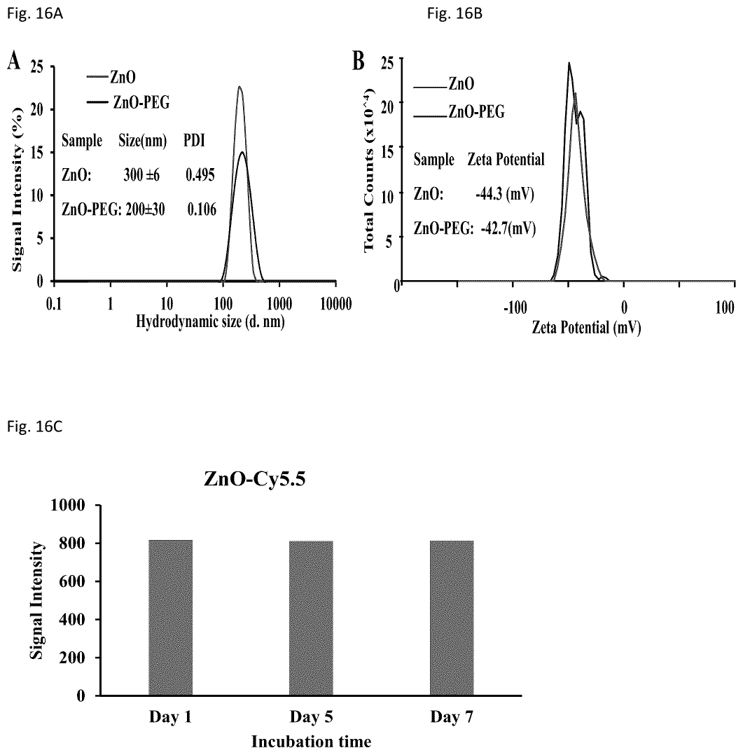

Next the temperature stabilization conferred to RNA by binding ZnO—NP was investigated by differential scanning calorimetry (DSC). DSC can be used to estimate the melting temperature (Tₘ) for RNA. Further circular dichroism (CD) spectroscopy can be used to measure the structural stability imparted by binding to ZnO—NP. DSC and CD data demonstrated the temperature-stability and structural stability which occurs for ZnO NP interaction to RNA are shown (FIG. 15).

FIG. 15 shows ZnO—NP complexation to poly I:C increases its melting temperature from 63.9-64.7° C. by approximately five to six degrees (Tₘ=70.1-71.1° C.). The presence of a biocompatible concentration of ZnO—NP (20 microgr/ml) thus caused an increase in RNA temperature-stabilization as shown in the left panel. The CD data shown in FIG. 15 right panel are consistent with the DSC data, the typical two peak CD pattern for poly I and poly C which is known to form an A-form RNA double helix is actually stabilized with peak enhancement when ZnO NP is complexed to poly I:C (right panel). Structural stability was a function of the stoichiometric ratio of poly I:C:ZnO—NP. Given the results shown in the top panels of FIG. 15 the accelerated stability of the ZnO-protamine-RNA formulation was investigated next. The preparations were sedimented from alcohol, air dried and then re-suspended in phosphate buffered saline (PBS) and stored for two weeks at 30, 40 or 50° C., versus material that was left out on the bench (20° C.) or in the refrigerator (4° C.) for 2 weeks. Afterwards a 100 microliter aliquot of each sample was removed, reprecipitated from alcohol, the RNA was eluted from the particles and analyzed again by RNA agarose gel electrophoresis (RAGE) As shown in the bottom panel to FIG. 15, the RNA band co-migrating with control could be observed for all temperature storage conditions. Two different sizes of ZnO NP were tested, 14 nm versus 100 nm ZnO—NP and the RNA band staining intensity was slightly higher for 14 nm in comparison to 100 nm. As expected some degree of band broadening and some smearing was seen, none-the-less the presence of RNA was clearly observed even after two week storage at the higher temperatures. Importantly, this stability and retention of RNA integrity was here maintained by liquid formulations, even after high temperature exposure for two weeks. No lyophilization, concentration or dry powder step was required in order to demonstrate this stability.

The biological activity of the ZnO-protamine-mRNA was assessed next either by expression or delivery experiments. For expression, in vitro translation experiments were next conducted using Luciferase (Luc) mRNA in the presence of rabbit reticulosate assay. Three experiments were run for control and two experiments for the ZnO-protamine-mRNA containing samples in the presence of high (1.0 mg/ml), medium (0.1 mg/ml) or low (0.01 mg/ml) concentrations of protamine. In addition to mRNA expression, mRNA delivery was assessed by complexing green fluorescent protein (GFP) mRNA to the protamine coated and/or adding an additional layer of protamine on top to promote delivery and expression in cells (FIG. 11):

As shown in FIG. 11 left panel, although some variability was observed with two different batches of Luc mRNA used, both batches of ZnO-protamine-RNA formulations showed expression activity. There was no apparent relationship between protamine and translation as it is thought that the ribosome will be able to outcompete protamine for the mRNA (unpublished data). Delivery into cells by the protamine (P) coated ZnO was in the order ZnO-P-RNA-P>ZnO-P-RNA>ZnO-P-RNA, when using either the GFP protein itself or GFP-encoding mRNA as measured by relative fluorescence per well.

### CONCLUSIONS

Lipid nanoparticles were first off the block, receiving clinical approval for COVID-19 mRNA vaccination. However inorganic nanoparticles can bind and deliver RNA into cells also.

Although inorganic NP are unable to internalize RNA, bound RNAs can still be stabilized, resist hydrolysis and temperature-induced degradation as shown here by RAGE, DSC, and CD analysis. Further protamine condensation has the additional advantage of being to increase payload of RNA and supports robust translation activity. Although the payload may be less, the advantage of RNA loading with LL-37 may confer additional antiviral activity and immuno-potentiation. Overall, the data shown here support inorganic NP, particularly zinc-based compositions such as ZnO or possibly others, for the stabilization and delivery mRNA, particularly in combination with cell penetrating or antiviral peptides.

### Example 5

### Materials and Methods

Nanomaterials and Reagents: All nanoparticles used were obtained pure from Sigma-Aldrich or PlasmaChem GmbH (Berlin, Germany), except for MnZnSe was provided by Dr. Emily McLaurin formerly in the Department of Chemistry Kansas State University, NiZnO was provided by Dr. Garry Glaspell US Army Corps of Engineers, Cobalt Zinc Oxide (CoZnO) cobalt ferrite PMC were synthesized by Dr. KC Ghosh's laboratory (Missouri State University). Poly inosinic:poly cytidylic acid [poly(I:C)] was obtained from Sigma-Aldrich (Cat #P958250MG). Cy5.5-labelled SSO (sequence: 3-CCUCUUACCUCAGUUACA-5) (SEQ ID NO. 1) was obtained from Trilink Biotechnologies. Clinical-grade LL-37 peptide (SEQ ID NO. 2) was obtained from our collaborator Dr. Cheng Kao Indiana University. NIH3T3 and A375 cells for cytotoxicity studies were obtained from ATCC. All NP and RNA were precipitated from 70% alcohol/H20 washed once with 100% alcohol, air dried in the biosafety cabinet prior to RNA and protein complexation, cell or animal administration. Copper (Cu) was purchased from PlasmaChem 10-100 nm in size. 50/50% Nickel Zinc Oxide (NiZnO), and 10/90% NiZnO were synthesized by Dr. G Glaspell's laboratory (Virginia Commonwealth University). Cobalt Oxide (Co3O4) and Nickel Oxide (NiO) pure powders were physically mixed, heated to a flux, allowed to cool in an oxygen purged atmosphere and jet ball milled to nanoscale confirmed by transmission electron microscopy and nanoparticle tracking analysis. The NPs were washed with double-distilled water, 70% ethanol/water, ethanol, and were stored dry prior to use. Costar (Corning, NY, USA) 96-well black, clear bottom assay plates were used for the assays. Luciferase enzyme (Photinus pyralis, ≥10×1010 (units/mg protein) was obtained from Sigma Aldrich and diluted it to a 0.2% solution [1:500 dilution with PBS buffer]. PBS buffer at 10× concentration was diluted to a 10% solution with de-ionized water [ddH20]. Luciferase enzyme substrate buffer (ATP, Mg) was diluted to a 1:1 vol/vol ratio with PBS buffer. Obtained β-Galactosidase (β-Gal) from Aspergillus oryzae was obtained from Sigma Aldrich (≥8.0 units/mg solid, Louis, MI, USA) and was diluted to a 1 mg/kg solution in spectral grade H20. B-Gal substrate, Resorufin β-D-galactopyranoside was purchased in a 10 g vial from Marker Gene Technologies (Eugene, OR, USA) and was diluted down into ten 10 mg/kg aliquots in spectral grade H20 and re-suspended into a 1 mg/kg solution for experimentation. Fluorescence emission, excitation and intensities were determined by Spectramax Paradigm.

Animal Model Imaging: Animal procedures followed approved IACUC 4064.1. Female 6 week old BALB/C Nu/Nu mice were obtained from Charles River and allowed to acclimate for several weeks prior to the experiment. These were anesthetized using oxygen/isoflurane prior to administration with treatment and bioimaging. Animals were intravenously administered with 100 μl of with PBS or ZnO NP or ZnO NP-Cy5.5 at the dose rate of 2 mg/kg body weight. Imaging was done using a Pearl® Trilogy Small Animal Imaging System (LI-COR Biosciences, USA) immediately before and after PBS administration, 5 days after ZnO NP administration and at regular time points until 6 h after ZnO NP-Cy5.5 administration. After the defined time points, animals were euthanized under anesthesia. Nanoparticle analysis was provided by the Nanotechnology Innovation Center Kansas State.

ICP-MS: For this procedure, tissues were collected from mice treated with, (1) PBS alone (n=1), (2) ZnO NP (n=2), and (3) ZnO NP-Cy5.5 (n=2). The concentration of zinc (Zn) in the mouse tissues was determined using ICP-MS analysis following the standard protocol. In brief, tissues were digested using 2 ml of 70% nitric acid (HN03) for liver and 1 ml for brain, heart, lungs, spleen and kidneys. The digestion was performed in SC154 HotBlock® (Environmental Express, USA) at 90° C. overnight. Following overnight digestion, all the tissue digests were diluted by addition of 9 ml deionized water. The diluted digests were further diluted by combining 1 ml of the digest with 4 ml of 2% HNO3 and filtered using 0.2 μm filter. Zinc concentration was measured on a PerkinElmer NexION® 350D ICP-MS.

Tissues were collected from mice treated with, (1) PBS alone (n=1), (2) ZnO NP (n=2), and (3) ZnO-Cy5.5 NP (n=2). Fluorescence was measured in the following tissues:
(1) PBS treated: Liver and Kidney
(2) ZnO NP treated: a. Mouse 1: Liver and kidneys b. Mouse 2: brain, heart, lungs, spleen and kidneys
(3) ZnO NP-Cy5.5 treated: brain, heart, lungs, spleen and kidneys from both mice

From the tissues collected, a portion was cut off, weighed and homogenized using SONICS VCX Vibra 130 Tissue Sonicator (PRO Scientific Inc.) at an amplitude of 50 at a pulse rate of 10 s(on) and 5 s (off) for 20 minutes. From the homogenate, 200 μl was transferred to a 96-well plate and fluorescence was measured using SpectraMax® i3× multimode microplate reader (Molecular Devices, California, USA). Excitation and emission wavelengths used were 660 nm and 695 nm respectively. PBS was used as the blank. The experiment was done in triplicates. Histopathology and hematology analyses were performed at the Veterinary Medical Diagnostic Laboratory, KSU. The remainder of the collected tissues were fixed in 10% neutral buffered formalin. Sections of fixed tissue were routinely processed on a Sakura Tissue-TEK VIP 6 Processor prior to paraffin embedding. Slides were cut at 4 μm and routinely stained with hematoxilin and eosin on a Leica Autostainer XL ST5010. Representative images at 10× magnification were captured on an Olympus LC20 camera mounted on an Olympus BX53F2 light microscope with CellSens (Olympus Corporation).

### Cellular Delivery and Nanomaterial Binding Assays:

3D Spheroid Culture of Caco2 cells: Human Caco2 cells (ATCC®, passage 30) were seeded onto a 35 mm sterile glass-bottomed cell culture dish (FluoroDish™-World Precision Instruments) to form 3D spheroids in a thin layer of 10% Matrigel (Corning® Matrigel® Basement Membrane Matrix, LDEV-free). Culture medium was comprised of 1×Minimum Essential Media (MEM, L-glutamine free), 10% Fetal bovine serum, 1% L-glutamine, and 1% Pen/Strep. Caco2 spheroids were in culture for approximately 24-hours prior to imaging. Delivery of ZnO-PEG-Cy5.5 (ZnOCy5.5) nanoparticles (NPs) to human Caco2 spheroids: Immediately prior to delivery, ZnOCy5.5 NPs were diluted to a stock concentration in Ham's F12 medium (160 μg/mL) sonicated for 60 seconds at room temperature (Fisher Scientific 60 Sonic Dismembrator Model F60 Cell Disrupter). Caco2 Matrigel-embedded spheroids were exposed to 20 μg/mL of ZnOCy5.5 NPs in culture media overnight in a 5% CO2 humidified incubator at 37° C. Post-treatment (16-hours), the Caco2 cells were evaluated for NP uptake by confocal microscopy using a FluoView FV1000 Inverted Confocal Microscope. Images were acquired with a 60× oil objective and the following laser settings (CHS1: Cy5.5, 795v, 1× Gain, 7% offset, laser 635 (1%), TD1:235v, 1× Gain, 0% offset, laser 488 (11%). Images were imported into SlideBook Version 5.0 (SB 5.0.0.14 5/13/2010) for presentation.

Delivery: B16F10 cells were incubated with cy5.5-ASO control or complexes with cy5.5-ZnO—NP cy5.5-ASO:Co3O4 NP incubated for 24 h, rinsed with PBS and imaged by confocal microscopy.

Similarly, NIH3T3 cells were treated with Cas9-GFP fusion protein or the MgO-NP control or MgO-Cas9 NP and imaged by fluorescence microscopy. B16F10 melanoma cells were incubated with cy5.5-ASO control or the complexes with ZnO, Co3O4 or NiO NP and 24 h later, rinsed with PBS, trypsinized and analyzed for cellular fluorescence by flow cytometry (K-State VDL core lab). B16F10 melanoma spheroids were allowed to establish for several days and treated with cy5.5-poly I:C viral mimetic RNA, the ZnO NP or cobalt (Co/ZnO) or nickel (Ni/ZnO) composites and imaged by fluorescence microscopy. Engineered human melanoma cells we previously reported under the control of an ASO inducible luciferase expression and delivery in the 5 replicate wells quantified by relative luminescence per well.

Mouse tumor was isolated at the time of metastasis, 20 mg samples lysed, the proteins extracted (2-3 tumors were pooled) and standardized to A280 (Molecular Devices Spectramax i3×, Sunnyvale, CA, USA). Slides were incubated with Cy3-Streptavidin (Sigma Aldrich, St. Louis, MO, USA), dried by centrifugation and stored under dark conditions and imaged using Molecular Devices Genepix 4000B (Sunnyvale, CA, USA). Nanobio interaction was confirmed by CD, FT-IR and zeta potential and payload estimated as previously described. The molecular dynamics simulation was performed with the peptide in the water far above the surface and observed the adsorption process. Fluorescent microscopy was conducted on an Olympus IX73 within poly-D-lysine coated 8 chamber slide, cells inoculum density (5×10⁴) after o/n adherence exposed to 20 ug/ml NP with maximal payload of Cas9-GFP (Applied Biological Materials Inc. Richmond, BC, Canada) or cy5.5-SSO versus cy5.5-SSO control (200 nM) assayed in the Texas Red/Rhodamine filter/channel. 3-D spheroids were formed within Insphero Corp plates, 48 h later the media was changed with NP as above containing Rhoda-poly I:C or stained with Invitrogen live/dead stain and imaged in the bio-imager (Licor Pearl Trilogy) or on the fluorescence microscope respectively.

Bioluminescent/fluorescent Readings: Bio-luminescence and fluorescent readings were taken on PerkinElmer (Caliper) LifeSciences IVIS Lumina II imager. Fluorescent readings were set to sixty second exposure, medium binning, 1 F/stop, dsRed emission filter, and intensity viewed through Rainbow setting. Dose-response assay utilized NPs that elicited high relative light units (RLUs) in earlier experiments for further analysis of the effects of increasing NP dose when incubated with β-Gal. Co3O4, ZnO, NiO, MgO, 10% NiZnO, 50% NiZnO, 95% CoZnO, and 98% CoZnO nanomaterials were incubated with β-Gal in a 2:1 enzyme:substrate ratio (200 and 100 μg/ml respectively at a NP concentration of 1, 2, 5, 10, 50, 100, 200 and 400 μg/ml dose. Time course measurements looking at enzyme:nanoparticle interaction were taken at 0, 10, 30, and 60 minutes. Dose-dependent assay utilized 1 mg of each NP was weighed out on a XS204 Mettler Toledo (Columbus, OH, USA) analytical balance, placed into Eppendorf tubes, and made into a 1 mg/ml suspension with PBS buffer stock solution. Each well had a total volume of 200 μl. Bio-fluorescence Detection was performed by the SpectraMax i3× by Molecular Devices (San Jose, CA, USA) at 1, 10, 30, and 60 minutes.

Ex-Vivo Bio-imaging: Mouse specimens were provided by Comparative Medicine Group. Lung sections were removed and were evenly divided into 2 sections. Ex-vivo imaging was performed in the Pearl® Trilogy Bioimaging system. 1 mg/ml of MnZnSe was diluted to a 1:3 with HPLC water and injected into individual sample and then imaged under white light, 700, and 800 nm filter, with 85 μm resolution and “0” focus. Increasing volumes (μl) were injected (1-20) with increasing fluorescent output. Tissue slurry/homogenate preparation consisted of heart, liver, kidney, brain, spleen, and lung from three different mice. Tissues were weighed on the XS204 Mettler Toledo (Columbus, OH, USA) analytical balance. Sectioned samples 100 mg per ml were then placed in sterile 10% PBS buffer, and homogenized via Vibra-Cell Processor VCX 130 (Newton, CT, USA) for 2 minutes, with 10 second pulses and 5 seconds rest. Slurry composition contained stromal tissue homogenized in with the sample (liver and kidney); homogenate composition had stroma removed via centrifugation and removal of supernatant to a separate tube (lung, heart, small intestine, liver, kidney and spleen).

### 2-D Fds Characterization:

To obtain excitation, emission, and intensity data Molecular Devices Spectramax i3x spectrophotometer was used. The microplate is scanned without the lid using the Molecular Devices Spectramax i3x spectrophotometer utilizing the Spectral Optimization Wizard that is included in the Softmax Pro 6.4.2 accompaniment software (Sunnyvale, CA, USA). The device was set to read the fluorescence endpoints of unknown wavelengths. The photomultiplier (PMT) gain was set to high, flashes per read was six, and wavelength increment was 5 nm. Before the first read, the microplate is shaken at medium intensity in a linear mode. The microplate was read from the top at a height of 1 mm. The range of excitation and emission wavelengths was set to 250-830 nm and 270-850 nm, respectively.

PMC's (1 mg/ml) were spiked into tissue slurry/homogenate and then placed into BRAND@ 96-well black bottomed plates (CAT #781668). 200 μl of PMC/tissue slurry/homogenate was assayed via SpectraMax i3x by Molecular Devices (San Jose, CA, USA) for 2-Dimensional Fluorescence Difference Spectroscopy (2D-FDS). For the enzyme:NP conjugate, Luc was added (10 μl) with 200 μl of NP and spun down at 140 RPM for one minute and re-suspended and added to the microtiter plate. The wavelength settings were set to unknown, spin before read, and optimization settings were set at excitation (250-830 nm) and emission (270-850 nm).

### MTT Assay

For cytotoxicity (MTT) assay, NIH3T3 fibroblast cells were seeded on a 96-well plate with 5,000 cells/well and allowed to grow for 24 h in DMEM with 10% FBS and 1% penicillin/streptomycin. After 24 h, the medium was replaced with PMCs (5% NiZnO or 5% MgZnO) dissolved in DMEM at 10, 20 and 25 μg/ml. Each treatment was tested on four wells. Four wells containing DMEM alone served as the blank and four wells with untreated cells served as the control. The cells were monitored daily for visible cytotoxicity using a light microscope. After 24/48/72/96 h with the treatment, the treatment was removed. All the wells were rinsed once with PBS, followed by the addition of 110 μL of 1:20 mix of MTT: indicator-free DMEM. After incubating the plate for 5 h at 37° C., 85 μL of the mixture was removed from each well and 75 μL of dimethyl sulfoxide was added to solubilize the crystals. The plate was then kept at 37° C. in an orbital shaker with 175 revolutions/minute. After 15 minutes on the orbital shaker, the plate was then read on a Synergy H1 Hybrid MultiMode Microplate Reader for absorbance at a wavelength of 562 nm.

### Tissue Uptake and Cell Delivery

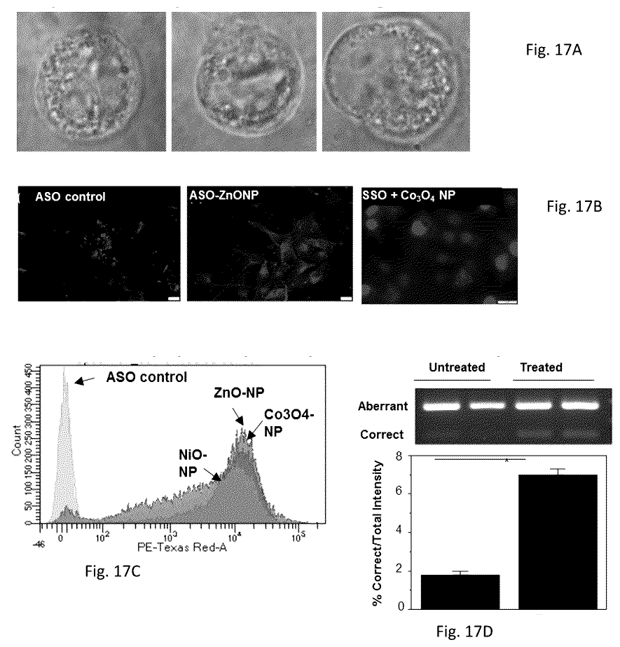

Robust uptake of the cy5.5-ZnO NP could be seen into 3-D cultures by confocal fluorescence microsopy. This experiment was conducted in triplicate for CACO2 gut organoids growing on matrigel and the nanoparticle very clearly distributed readily into the tissue. Antisense oligomer (ASO) have been clinically approved to treat rare disease and we designed an ASO to target the cryptic splice site associated with melanoma drug resistance. ZnO NP as well as cobalt oxide (Co₃O₄) NP increased cy5.5-ASO uptake into melanoma cells, indeed the Co-NP appeared to provide for functional nuclear delivery which was confirmed in our functional assay (data not shown). ASO delivery by ZnO, Co or NiO NP was confirmed by flow cytometry and the molecular mechanism was as expected correction of the aberrant spliced Ras binding domain (RBD) as shown by RT-PCR (FIG. 17 A-D).

As can be seen in FIG. 17, cy5.5-ZnO is readily taken into 3-D organoid tissues (FIG. 17A) and increases the uptake and intracellular distribution of cy5.5-ASO into melanoma cells (FIG. 17B) as shown by confocal fluorescence microscopy with apparent nuclear ASO location for Co₃O₄ NP. Increased ASO uptake stimulated by the NP delivery was confirmed by flow cytometry, where the ZnO, Co₃O₄ or NiO NP complexed to cy5.5-ASO had a 3-log order shift in cellular fluorescence compared to cy5.5-ASO oligo only control (FIG. 17C). The functional affect of NP delivery of ASO was shown by RT-PCR with the corrected transcript appearing when treated with NP-ASO complex versus untreated controls (FIG. 17D). With the increased cell and tissue uptake of ZnO, cell association and nuclear delivery of Ni and Co materials these data suggested the possibility of synthesizing composites combining these biometals as described next

### Zn-Based Physiometacomposites (PMC).

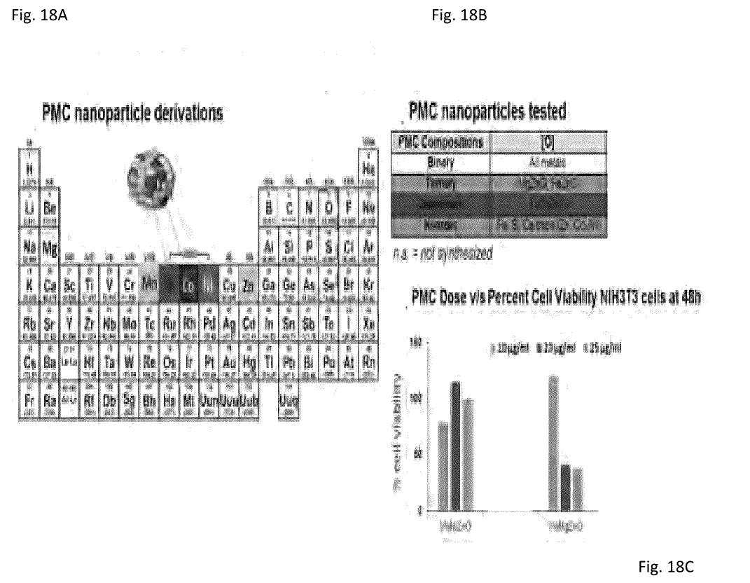

Metamaterials or nanoparticle composites have unique physico-chemical properties but their delivery, antiviral and anticancer activity has never been explored. An initial library containing nickel (Ni) and cobalt (Co) in the context of precious metals was synthesized by the Mirkin group. Recently we described the synthesis of cobalt-zinc oxide (CoZnO) and nickel-zinc oxide (NiZnO) and here the zinc-based series was expanded to include MgZnO, FeZnO, FeCoZnO, and Hi-order PMC compositions with Fe, Si, Ca trace (Zn, Co, Ni). Nanoscale and nanorod shape of these materials was confirmed by transmission electron microscopy (TEM) and nanoparticle tracking analysis. The biocompatibility of these PMC nanomaterials was tested at 10, 20 and 25 ug/ml with highly sensitive non-transformed NIH3T3 cells (FIG. 18C).

Importantly, FIG. 18 shows the outstanding biocompatibility of the Zn-based PMC nanoscale materials. Very little cytotoxicity is seen after 48 hour continuous exposure to untransformed highly sensitive NIH3T3 cells at 10 microgr/ml concentration (FIG. 18C). The MgZnO material was relatively toxic at the two higher doses, either 20 or 25 microgr/ml whereas the NiZnO was intermediate and better tolerated.

### 3-D Tumor Spheroid Imaging and Inhibition

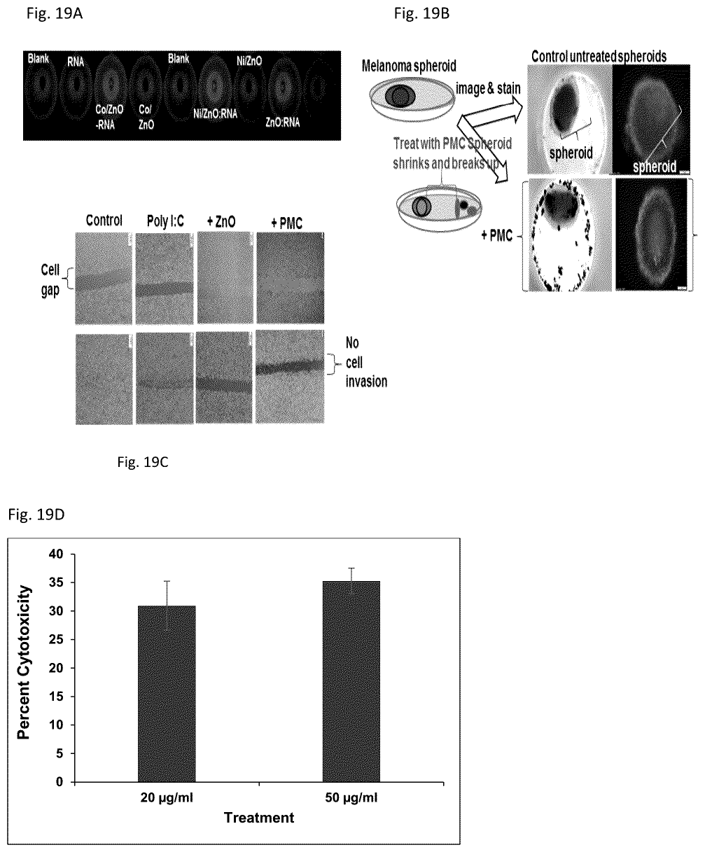

Previously poly I:C RNA had been combined with a iron-zinc oxide composite shown to potentiate its anticancer activity. Here we cy5.5-labeled poly I:C and showed that the PMC were able to label 3-D tumor spheroids imaged in the bio-imager far-red/NIR range. Intravital staining of the spheroids clearly showed an increase in the kill zone when treated with 20 microgr/ml PMC nanoparticle consistent with antitumor activity indeed the dense inner layer of cells which appears dark on light microscope examination breaks apart after nanoparticle treatment. Both ZnO and CoZnO PMC were able to inhibit drug-resistant melanoma (B16F10) cell invasion in the classic scratch assay (FIG. 19).

As shown in FIG. 19, PMC are able to load and label 3-D spheroids with cy5.5-poly I:C (FIG. 19A). 3-D tumor spheroids treated with PMC break apart and many of the cells within the dense interior dye as shown by green/red intravtial staining (FIG. 19B). The PMCs or ZnO NP control also inhibit cancer cell invasion in the scratch assay (FIG. 19C). These data suggest the anticancer or antitumor activity of ZnO NP and second-generation PMC materials which was tested next.

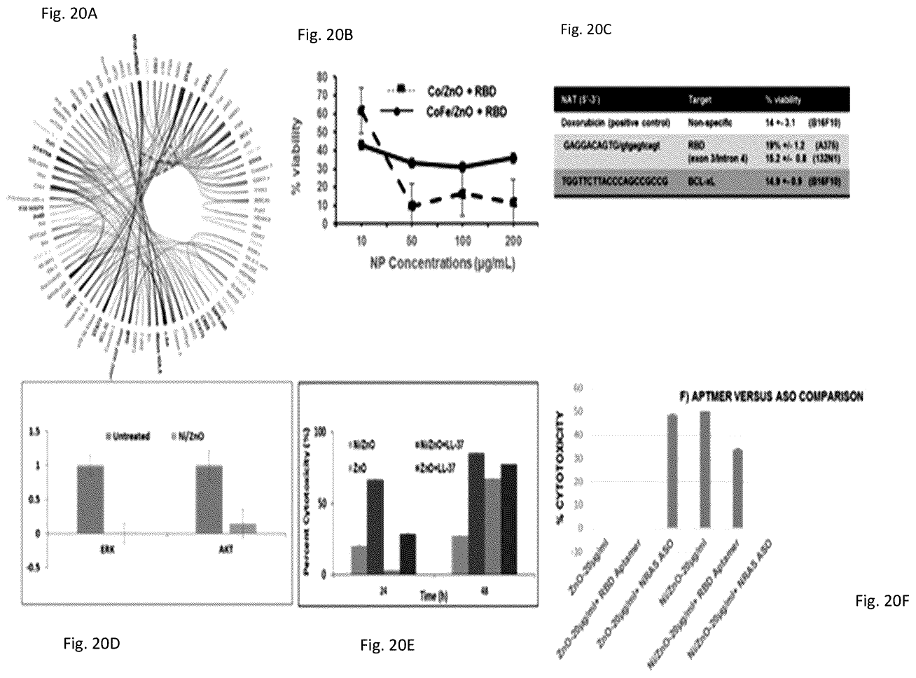

ANTICANCER ACTIVITY. Anticancer activity of the PMC derivations and their ASO and aptamer complexes was tested next. First melanoma tumor at the time of metastasis was harvested and subjected to high throughput proteomics analysis confirming the import of proteins in the RAS/ERK/AKT pathways and secondarily BCL apoptosis pathways. To address this melanoma or model glioblastoma line (132N1) were treated with NP delivered RBD decoy protein interference or ASO targeting RBD or BCL-xL demonstrating outstanding inhibition (FIG. 20).

As shown in FIG. 20, proteomics analysis revealed multiple proteins in the RAS/ERK/AKT associated with metastatic melanoma (FIGS. 20A and 20C). This could be targeted wither by PMC delivery of RBD decoy as a protein interference approach for CoZnO or CoFeZnO ternary or quaternary composites (FIG. 20B), or by ASO targeting RBD or BCL-xL. Dogs are considered an excellent comparative oncology model for melanoma especially for rarer drug resistant forms, the canine mucosal melanoma are activated in the ERK/AKT pathway and are considered an excellent preclinical model for nanomedicine testing. These cells could be greatly inhibited by NiZnO even at early timepoints, but ironically in concert with RBD targeted ASO or aptamer no enhanced anticancer activity was seen, except for ZnO (FIG. 20E).

### Antiviral Activity

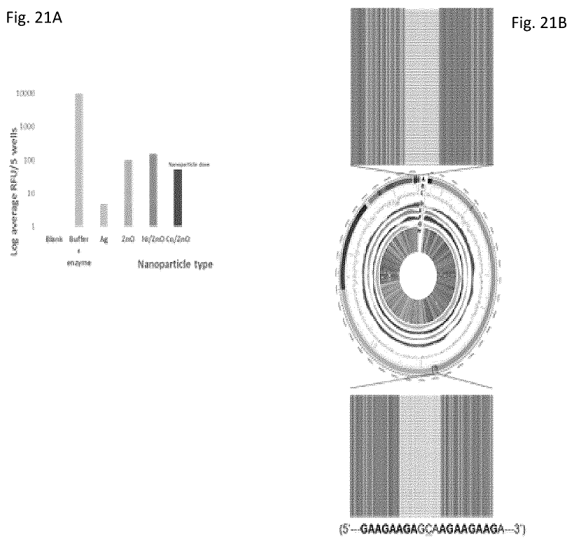

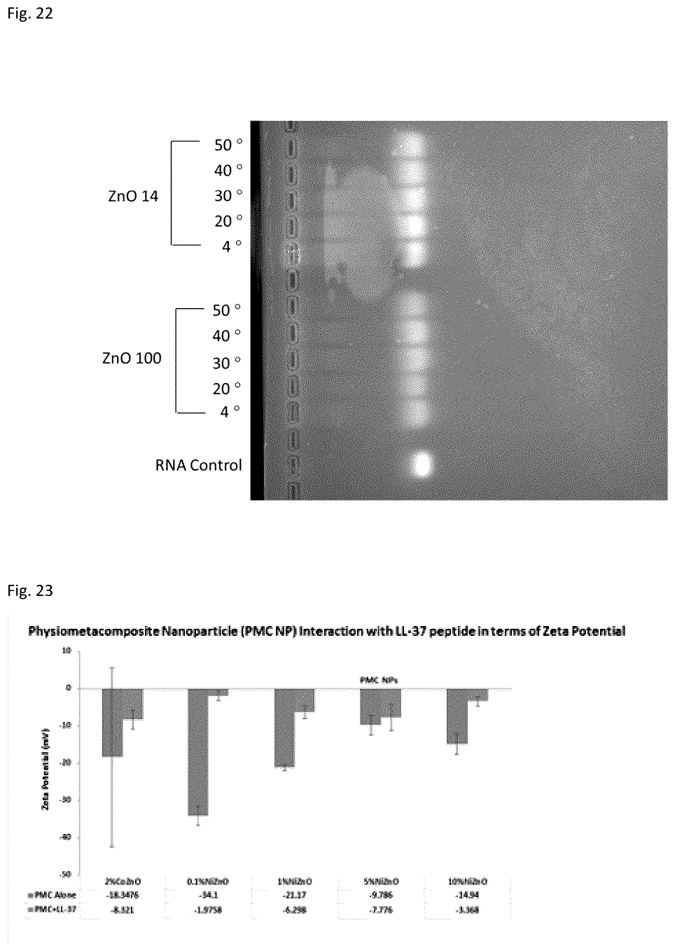

Antiviral activity: We next tested the enzyme inhibition, antiviral activity, and the design of ASO targeting putative conserved regulatory sites in SARS-COV-2 (FIGS. 21A and 21B).

As shown in FIG. 21, the antibacterial activity of ZnO NP has been correlated to b-Gal enzyme inhibition, and such inhibition was also shown with this experiment (data not shown). Finally we performed GWAS analysis on NSARS-CoV-2 RNA identifying the two most conserved sites (98-99%) in the 3′UTR and a partial homupurinic palindromic putative regulatory site for ASO targeting (FIG. 21B)

In conclusion these data support the further preclinical evaluation of the Zn-based physiometacomposite (PMC) nanomaterials for imaging and treatment of cancer and infectious disease. Especially for certain rarer drug-resistant forms of cancer such as mucosal melanoma and other ERK/AKT-activated metastatic forms where canine represents an excellent comparative oncology model for pre-clinical testing of protein and RNA-based nanomedicines. Here, Zn-based conjugates, either ZnO NP or the cy5.5 derivative are shown to distribute into liver, kidney, lung, spleen and brain. 3-D culture experiments suggest these materials are well taken up by tissues and are able to deliver ASO into cells as shown by confocal microscopy and flow cytometry. At least at the 20 mg/kg dosage after a single intravenous administration no overt toxicity was seen in the two animals treated and assessed after 5 hours or 3 days, and in cell culture at least for 48 hours of continuous exposure normal cell controls such as the NIH3T3 standard cells tested displayed very little cytotoxicity up to 25 microgr/ml, and in some cases cells could be treated for much longer up to 3 or even 4 days with 98-99% viability (data not shown). PMC RBD or RBD targeted ASO or aptamer showed dose-dependent cancer cell inhibition and could be used to inhibit drug-resistant rarer forms of melanoma such as canine mucosal melanoma (M5) line used here. These data overall support the evaluation of PMC conjugates of Ras-targeted ASO, aptamer and sequences targeting NSARS-COV-2 as a new anticancer/antiviral approach.

Zn-based physiometacomposite nanoparticles are biocompatible and indigenously fluorescent. Importantly PMC have antiviral activity and can achieve significant RNA payloads and impart structural retention and temperature stabilization to RNA. PMC materials are thus of interest for pre-clinical applications of RNA vaccines and RNA-based therapeutics.

What is claimed is:

1. A nanoparticle complex comprising:
a nanoparticle selected from the group consisting of ZnO, Fe₃O₄, Ag, Cu, MSN, CNT, and any combination thereof; and
at least one component selected from the group consisting of RBD (must define), cobalt, nickel, a peptide, an antisense oligomer, an aptamer, protamine, yeast, poly I:C, manganese, iron, magnesium, cobalt ferrite, and any combination thereof.

2. The nanoparticle complex of claim 1, wherein the peptide is SEQ ID NO. 2.

3. The nanoparticle complex of claim 1, wherein the at least one component is positioned on the surface of the nanoparticle.

4. The nanoparticle complex of claim 1, wherein the nanoparticle is doped with the at least one component.

5. The nanoparticle complex of claim 1, wherein the nanoparticle is complexed with mRNA or RNA.

6. A method of stabilizing mRNA or RNA comprising the step of complexing the mRNA or RNA with the composition of claim 1, wherein the stabilization of the mRNA or RNA is determined by increased resistance to degradation.

7. The method of claim 6, wherein the degradation is caused by increased temperature.

8. The method of claim 6, wherein the extent and/or rate of degradation is decreased by at least 10% in comparison to mRNA or RNA that is not complexed with the composition of claim 1.

9. The method of claim 6, wherein the stabilization of the mRNA or RNA is determined by a comparison of the structure of mRNA or RNA complexed with the composition of claim 1 with mRNA or RNA that is not complexed with the composition of claim 1.

10. A method of treating cancer comprising the step of administering the composition of claim 1 to a subject in need thereof.

11. The method of claim 10, wherein the cancer is metastatic.

12. The method of claim 10, wherein the composition of claim 1 comprises a nanoparticle complexed with a peptide, RNA, or mRNA.

13. The method of claim 12, wherein the peptide is LL37.

14. The method of claim 12, wherein the peptide, mRNA, or RNA targets the RAS binding domain (RBD), RAS interacting protein, or a downstream effector selected from the group consisting of ERK, AKT, and any combination thereof.

15. The method of claim 10, wherein the composition of claim 1 is administered systemically.

16. A method of treating a virus or microbial infection comprising the step of administering the composition of claim 1 to a subject in need thereof.


SEQUENCE LISTING (5 sequences)

SEQ ID NO: 1 - RNA, 18 nt, Artificial Sequence
  Probe
ccucuuaccucaguuaca

SEQ ID NO: 2 - protein, 37 aa, Homo sapiens
LLGDFFRKSKEKIGKEFKRIVQRIKDFLRNLVPRTES

SEQ ID NO: 3 - DNA, 10 bp, Artificial Sequence
  Probe Targeting Exon 3 of RBD
gaggacagtg

SEQ ID NO: 4 - DNA, 10 bp, Artificial Sequence
  Probe Targeting Intron 4 of RBD
gtgagtcagt

SEQ ID NO: 5 - DNA, 20 bp, Artificial Sequence
  Probe Targeting BCL-xl
tggttcttacccagccgccg